(19) United States
(12) Patent Application Publication
(10) Pub. No.: US 2023/0416317 A1
(43) Pub. Date: Dec. 28, 2023

(54) METHODS OF TREATING OR PREVENTING NEUROLOGICAL DISORDERS USING ZPR1

(71) Applicant: Texas Tech University System, Lubbock, TX (US)

(72) Inventor: Laxman Gangwani, El Paso, TX (US)

(73) Assignee: Texas Tech University System, Lubbock, TX (US)

(21) Appl. No.: 18/038,177

(22) PCT Filed: Nov. 22, 2021
(86) PCT No.: PCT/US2021/060258

§ 371 Date: May 22, 2023

(51) Int. Cl. C07K 14/47; A61P 25/28

(52) U.S. Cl. CPC C07K 14/4702; C07K 2319/60; A61P 25/28

(57) ABSTRACT

Embodiments of the present disclosure pertain to methods of treating or preventing a disorder in a subject by administering to the subject a composition that includes one or more of the following active components: a zinc finger protein ZPR1 (ZPR1), an analog thereof, a homolog thereof, a derivative thereof, or combinations thereof; an enhancer of expression of ZPR1, an analog thereof, a homolog thereof, or combinations thereof; nucleotides encoding ZPR1, an analog thereof, a homolog thereof, a derivative thereof, or combinations thereof; or combinations thereof. The disorder to be treated or prevented may be caused by at least one mutation in the Senataxin (SETX) gene, a downregulation of SETX protein levels, or may include a neurodegenerative disease or disorder. Further embodiments pertain to the aforementioned compositions, which may be suitable for use in treating or preventing one or more of the aforementioned disorders in a subject.

### CROSS-REFERENCE TO RELATED APPLICATIONS

This application claims priority to U.S. Provisional Patent Application No. 63/116,417, filed on Nov. 20, 2020. The entirety of the aforementioned application is incorporated herein by reference.

### STATEMENT REGARDING FEDERALLY SPONSORED RESEARCH

This invention was made with government support under R01 NS064224 and R01 NS115834 awarded by the National Institutes of Health. The government has certain rights in the invention.

### BACKGROUND

Many neurodegenerative disorders remain incurable with very limited treatment options. Numerous embodiments of the present disclosure address the aforementioned limitations.

### SUMMARY

In some embodiments, the present disclosure pertains to methods of treating or preventing a disorder in a subject by administering to the subject a composition that includes at least one active component. In some embodiments, the active component includes at least one of: (1) a zinc finger protein ZPR1 (ZPR1), an analog thereof, a homolog thereof, a derivative thereof, or combinations thereof; (2) an enhancer of expression of ZPR1, an analog thereof, a homolog thereof, or combinations thereof; (3) nucleotides encoding ZPR1, an analog thereof, a homolog thereof, a derivative thereof, or combinations thereof; or (4) combinations thereof.

In some embodiments, the disorder to be treated or prevented includes a disease or disorder caused by at least one mutation in the Senataxin (SETX) gene, a downregulation of SETX protein levels, or combinations thereof. In some embodiments, the disorder includes a neurodegenerative disease or disorder, such as amyotrophic lateral sclerosis 4 (ALS4), ataxia with oculomotor apraxia type 2 (AOA2), spinal muscular atrophy (SMA), autosomal dominant SMA (ADSMA), or combinations thereof.

Additional embodiments of the present disclosure pertain to the compositions of the present disclosure, which include at least one of the aforementioned active components. In some embodiments, the compositions of the present disclosure may be suitable for use in treating or preventing one or more of the aforementioned disorders in a subject by administering the composition to the subject.

### DESCRIPTION OF THE DRAWINGS

FIG. 1 illustrates a method of treating or preventing a disorder in a subject by administering the compositions of the present disclosure to the subject.

FIGS. 2A-F demonstrate that ZPR1 interacts with SETX and R-loops and facilitates SETX recruitment onto R-loops. FIGS. 2A-C show that ZPR1 and SETX physically interact and form complexes with R-loops. Immunoprecipitations (IPs) and GST pulldowns were examined by capillary-based automated western blot system. FIG. 2A shows IP of ZPR1 with antibody against ZPR1 (IP: ZPR1) from HeLa cell lysate followed by western blot (WB) analysis using antibody against SETX (WB: SETX), which demonstrate that SETX binds in vivo with ZPR1. FIG. 2B shows IP of SETX with antibody against SETX (IP: SETX) from COS-7 cells expressing recombinant ZPR1-GFP* followed by WB with antibody against GFP (WB: GFP) to detect fusion protein ZPR1-GFP. FIG. 2C is a GST pulldown assay showing recombinant GST-ZPR1 fusion protein pulls down SETX from HeLa cell lysate. FIGS. 2D-E show that ZPR1 interacts in vivo with R-loops and is part of SETX containing R-loop resolution complexes. IP of R-loops (IP: R-loops) were performed using monoclonal antibody (S9.6) against RNA-DNA hybrids from HeLa cell lysate followed by WB analysis. IP with S9.6 antibody shows co-immunoprecipitation (Co-IP) of SETX (FIG. 2D) and ZPR1 (FIG. 2E) with R-loops. FIG. 2F shows IP of R-loops with S9.6 antibody, illustrating ZPR1 knockdown (As-ZPR1) causes a decrease in SETX binding with R-loops.

FIGS. 3A-3G demonstrate that ZPR1 colocalizes with SETX in nuclear bodies (NBs) and its deficiency causes disruption of gems and Cajal bodies, downregulation of SETX and accumulation of R-loops. HeLa cells (Control) or transfected with 100 nM antisense oligonucleotides against human ZPR1 (As-ZPR1) or scrambled sequence oligo (Scramble) were fixed and stained with antibodies for immunofluorescence (IF) analysis. FIG. 3A illustrates quantification of SETX colocalization in sub-nuclear bodies (NBs)/cell (%), which is shown as a violin plot with median and interquartile range (Q1, median, Q3) (50 cells/group). SETX colocalization with ZPR1: ZPR1+SETX (72.73, 80.00, 100.00), SMN: SMN+SETX (32.89, 57.14, 80.83) and coilin: Coilin+SETX (24.31, 50.00. 68.75). FIGS. 3B-D show that ZPR1 knockdown causes downregulation of SETX. FIG. 3B shows immunoblots (IBs) of ZPR1, SETX and tubulin from cell lysates of Control, As-ZPR1 and Scramble transfected HeLa cells. FIG. 3C provides quantitative (mean±SEM, n=3) and statistical analysis (ANOVA) showing knockdown of ZPR1 levels to (22.44±3.01%, P=0.0002) decrease SETX levels to (44.41±8.20%, P=0.0036) compared to Control and Scramble. FIG. 3D shows that knockdown of ZPR1 mRNA expression to (34.05±7.21%, P=0.0007) causes downregulation of SETX mRNA expression to (47.76±9.72%, P=0.0061) compared to Control and Scramble. FIG. 3E shows quantitative analysis of nuclear R-loop IF intensity with NIH ImageJ software show ZPR1-deficient cells (As-ZPR1) accumulate R-loops (7.80±0.37-fold, P<0.0001) compared to Control and Scramble cells. R-loops nuclear intensity levels were quantified from three experiments (30 cells/group). FIGS. 3F-G shows quantitative mapping of R-loop accumulation throughout transcription of the human $-Actin (ACTB) and GAPDH genes. DNA-RNA immunoprecipitation (DRIP) was performed using S9.6 antibody and genomic DNA prepared from control, control+RNase H, ZPR1-deficient (As-ZPR1) and As-ZPR1+RNase H treated HeLa cells. DRIP and input DNA were used for qPCR analysis using specific primers pairs to amplify different regions of R-loop accumulation during transcription of the ACTB gene in (FIG. 3F) control, control+RNase H, As-ZPR1 and As-ZPR1+RNase H, and (FIG. 3G) the GAPDH gene in control, control+RNase H, As-ZPR1 and As-ZPR1+RNase H. Quantitative analysis (mean±SEM, n=3) shows ZPR1-deficiency causes ˜4-5-fold R-loop accumulation throughout transcription, including transcription start. Loss of R-loops with RNase H treatment shows specificity of DRIP analysis.

FIGS. 4A-F show that SETX deficiency causes disruption of ZPR1 positive NBs, gems and Cajal bodies and accumulation of R-loops during transcription. HeLa cells, untransfected (Control) or transfected with 100 nM siRNA against SETX (siSETX) and siRNA with scrambled sequence (Scramble), were fixed and stained with antibodies for immunofluorescence (IF) analysis. FIG. 4A shows immunoblots (IBs) of SETX, ZPR1 and tubulin from cell lysates of Control, siSETX and Scramble transfected HeLa cells. FIG. 4B provides a quantitation showing knockdown of SETX (siSETX) levels to (21.77±4.19%, P=0.0024) does not change significantly ZPR1 levels (89.97±8.37%, P=0.6447) compared to Control and Scramble. FIG. 4C shows knockdown of SETX mRNA expression to (25.80±3.61%, P=0.0007) does not significantly alter ZPR1 mRNA expression (81.83±6.61%, P=0.0872) compared to Control and Scramble. FIG. 4D provides a quantitative analysis of nuclear R-loop IF intensity with NIH ImageJ software showing SETX-deficient cells (siSETX) accumulate R-loops (3.85±0.39-fold, P=0.0002) compared to Control and Scramble cells. Quantitative mapping of R-loop accumulation throughout transcription of the β-Actin (ACTB) and GAPDH genes. DRIP was performed using S9.6 antibody and genomic DNA prepared from control, control+RNase H, SETX-deficient (siSETX) and siSETX+RNase H treated HeLa cells. DRIP and input DNA were used for qPCR analysis using specific primers pairs to amplify different regions of R-loop accumulation during transcription of the ACTB gene in (FIG. 4E) control, control+RNase H, siSETX and siSETX+RNase H (FIG. 4F) the GAPDH gene in control, control+RNase H, As-ZPR1 and siSETX+RNase H. Quantitative analysis (mean±SEM, n=3) of SETX-deficiency shows R-loop accumulation (˜2-3-fold) throughout transcription except at the start of transcription compared to control. Loss of R-loops with RNase H treatment shows specificity of DRIP analysis.

FIGS. 5A-5G show chronic low levels of ZPR1 impair assembly of RLRC in SMA. Cultured WI-38 (Normal) and primary fibroblast derived from SMA type I patients, GM03813 and GM09677 (SMA) that have homozygous deletion of the SMN1 gene, were used for IF, IP and IB analyses. FIG. 5A shows representative capillary-blot images of proteins. FIG. 5B provides quantitative analysis (mean±SEM, n=3, t-test, unpaired), showing that SMN1 mutation results in the low levels SMN in GM03813 (25.14±2.88%, P=0.0003) and GM09677 (23.06±1.92%, P=0.0002) SMA patient cells compared to normal cells. Chronic SMN-deficiency is known to cause splicing defects and alter expression of many genes. Analysis of core components of RLRC shows ZPR1 levels decreased to (45.03±6.46%, P=0.0045) in GM03813 and (46.18±11.15%, P=0.0148) in GM09677. SETX levels decreased to (44.27±3.25%, P=0.0002) in GM03813 and (48.62±4.17%, P=0.0006) in GM09677 compared to control. Chronic ZPR1-deficiency results in decrease of ZPR1 complexes with SETX and SMN in SMA compared to normal cells. IP of ZPR1 shows decrease in co-immunoprecipitation of (FIG. 5C) SETX and (FIG. 5D) SMN from SMA (GM03813, GM09677) compared to normal cells. FIG. 5E is an IP of SETX showing decrease in pulldown of SMN. IP of R-loops shows decrease in association of (FIG. 5F) SETX and (FIG. 5G) SMN with RNA-DNA hybrids in SMA cells compared to normal cells.

FIGS. 6A-L illustrate that ZPR1 rescues assembly of RLRC and averts accumulation of pathogenic R-loops in SMA. SMA patient primary fibroblast cell lines, GM03813 and GM09677 were transfected with phrGFP (GFP) or phrZPR1-GFP (ZPR1-GFP), fixed and stained with antibodies against SETX, SMN and R-loops for IF or cell lysates were prepared for IP and IB analyses. Ectopic ZPR1 expression elevates levels of SETX and SMN in SMA cells. Immunoblots show ZPR1 overexpression increases SETX and SMN levels in SMA patient cells (FIG. 6A) GM03813 and (FIG. 6C) GM09677. Quantitation (mean±SEM, n=3) and comparison of different protein levels in (FIG. 6B) GM03813+ZPR1-GFP cells show increase in levels of ZPR1 to (4.23±0.10-fold, P<0.0001), SETX (2.47±0.26-fold, P=0.0060), and SMN (4.48±0.51-fold, P=0.0026) compared to control GM03813+GFP cells. FIG. 6D illustrates that GM09677+ZPR1-GFP cells show increase in levels of ZPR1 (4.50±0.10-fold, P<0.0001), SETX (2.49±0.18-fold, P=0.0017) and SMN (4.99±0.32-fold, P=0.0005) compared to control GM09677+GFP cells. ZPR1 complementation decreases R-loop accumulation in SMA patient cells. FIG. 6E provides quantitative data (mean±SEM, n=4) showing R-loop accumulation in SMA cells (GM03813+GM09677) decreased to (29.21±5.69%, P=0.0004) in ZPR1-GFP⁺ compared to control cells. ZPR1 rescues defects in the assembly of core RLRC proteins, ZPR1 and SETX with R-loops in SMA patient cells. IP using S9.6 antibody against R-loops show association of ZPR1-GFP with R-loops in (FIG. 6F) SMA GM03813+ZPR1-GFP and (FIG. 6G) SMA GM09677+ZPR1-GFP cells. FIGS. 6H and 6I illustrate that IPs of R-loops from GM03813+ZPR1-GFP and GM09677+ZPR1-GFP show increased association of SMN with R-loops compared to SMA+GFP cells. FIGS. 6J and 6K illustrate that IPs of R-loops from GM03813+ZPR1-GFP and GM09677+ZPR1-GFP show increased association of SETX with R-loops compared to SMA+GFP cells. FIG. 6L provides quantitation and comparison of SETX in vivo association with R-loops between Normal, SMA+GFP and SMA+ZPR1-GFP showing that ZPR1 increases SETX binding from 31.66±5.30% (SMA+GFP) to 84.79±4.31% (P<0.0001) (SMA+ZPR1-GFP) with R-loops.

FIGS. 7A-7B illustrates that ZPR1 overexpression in vivo rescues DNA damage associated with R-loop accumulation and prevents degeneration of motor neurons in SMA. Primary spinal cord neurons were cultured from 7-day-old Normal, SMA and Z-SMA (SMA mice with ZPR1 overexpression under the control of mouse Rosa26 promoter) mice. Neurons differentiated in vitro for 12 days and stained with antibodies against neuron-specific β-tubulin-III (red), SMN, SETX, p-DNAPKcs, R-loops and 7H2AX, and IF was examined by confocal microscopy. FIG. 7A provides an immunoblot analysis of cultured primary spinal cord motor neurons from Normal, SMA and Z-SMA mice for detecting changes in levels of ZPR1, SMN, SETX, DNAPKcs, p-DNAPKcs, and DNA damage marker, 7H2AX. FIG. 7B provides quantitative immunoblot data as a bar graph. Statistical analysis (ANOVA) of IB data (mean±SEM, n=3 mice/group) from spinal cord neurons shows increase in ZPR1 levels (1.70±0.11-fold, P=0.0001) results in increase of SMN levels to (77.21±4.43%, P=0.0002), SETX (88.73±9.36%, P=0.0012), p-DNA-PKcs (81.90±6.92%, P=0.0037) and total DNAPKcs (87.98±7.31%, P=0.0018) leading to a marked decrease in 7H2AX levels from 306.80±10.26% to 120.30±5.77% (P=0.0002).

FIGS. 8A-K illustrate that the interaction of SETX with ZPR1 is disrupted in ALS4 patients and ZPR1 fails to recruit mutant SETX onto R-loops in ALS4. Mutational analysis shows that SETX L389S mutation, which causes autosomal dominant ALS4, disrupts interaction of SETX with ZPR1. FIG. 8A provides IP data, where COS7 cells were transfected with plasmids pDEST53 expressing GFP-hSETX (1-667) (WT) and GFP-hSETX (1-667) (L389S). IP was performed using anti-ZPR1 antibody followed by WB with anti-GFP to detect GFP-hSETX. FIG. 8B shows immunoblots of cell lysates from cells expressing GFP-hSETX (WT) and GFP-hSETX (L389S). To gain insight into the contribution of disruption of SETX-ZPR1 complexes in the pathogenesis of ALS4, Applicant used fibroblasts derived from normal subjects and ALS4 patients that have heterozygous SETX mutation L389S (SETX^{+/L389S}). Cultured control, Normal #1 and Normal #2, and ALS4 cell lines, ALS4 #3 and ALS4 #4 were used for IF, IB and IP analyses. FIG. 8C illustrates that the interaction of SETX with ZPR1 is disrupted in ALS4 patients. IP of ZPR1 shows decrease in co-immunoprecipitation of SETX from ALS4 patients compared to control (Normal) fibroblast. FIG. 8D provides quantification of SETX colocalization in normal cells (mean±SEM, n=50 cells/group), which shows higher colocalization with ZPR1 (94.33±5.36%) compared to SMN (69.67±5.28%) (gems) and Coilin (60.33±5.77%) (CBs). SETX colocalization in ALS4 cells also show higher colocalization with ZPR1 (49.33±7.25%) compared to SMN (34.17±6.18%) and Coilin (18.00±4.23%). The overall SETX colocalization is reduced by ˜48% (ZPR1, P<0.0001), ˜51% (SMN, P<0.0001) and ˜70% (coilin, P<0.0001) in ALS4 compared to normal cells. FIG. 8E shows quantitative (mean±SEM, n=4) and statistical (unpaired t-test) analysis of R-loop nuclear IF intensity in cells, showing fewer R-loops (53.72±9.85%, P=0.0125) in ALS4 patient cells (ALS4 #3+ALS4 #4) compared to control (Normal #1+Normal #2) cells. FIG. 8F provides IB analysis of protein ZPR1, SETX, SMN and tubulin in Normal #1 and Normal #2, and ALS4 #3 and ALS4 #4, patient cells. FIG. 8G provides quantitative analysis of protein levels (mean±SEM, n=3) showing SETX mutation did not significantly change the levels of ZPR1 (98.27±10.10%, P=0.8252), SMN (97.04±7.89%, P=0.9080) and SETX (93.67±7.27%, P=0.3903) in ALS4 compared to normal cells. FIG. 8H shows that SETX fails to associate in vivo with R-loops in ALS4. IP of R-loops shows marked decrease in SETX co-immunoprecipitation in ALS4 compared to control suggesting that ZPR1 fails to recruit mutant SETX to R-loops. FIG. 8I provides IPs of R-loops from Normal and ALS4 subjects infected with Ad5-GFP and Ad5-ZPR1-GFP, showing that the binding of ZPR1 with R-loops is unaffected under ALS4 disease conditions and supports the idea that ZPR1 interaction with SETX is critical for the recruitment of SETX onto R-loops. Quantitation of SETX co-immunoprecipitation (mean±SEM, n=4) with ZPR1 IP shows that (FIG. 8J) the levels of SETX binding with ZPR1 are reduced to (33.82±3.45%, P<0.0001) in ALS4 compared to control cells. FIG. 8K provides IP of R-loops, showing that SETX binding with R-loops reduced to (30.51±4.43%, P=0.0001) in ALS4 compared to control cells.

FIGS. 9A-H illustrate that modulation of ZPR1 levels regulates R-loop accumulation and rescues pathogenic R-loop phenotype in ALS4 patient cells. Quantitation (mean±SEM, n=3) of IBs from ALS4 #3 (FIGS. 9A and 9C) and ALS4 #4 (FIGS. 9B and 9D) control, As-ZPR1 and scramble samples show KD of ZPR1 to (19.69±2.13%, P=0.0001) in ALS4 #3 and (21.7±4.614%, P<0.0001) in ALS4 #4. ZPR1 KD decreases SETX level to (52.06±6.55%, P=0.0012) in ALS4 #3 and (53.28±4.33%, P=0.0002) in ALS4 #4. FIGS. 9E-H illustrate that ZPR1 overexpression improves in vivo association of SETX with R-loops in ALS4 patient cells. FIG. 9E shows immunoblots of ZPR1-GFP, SETX, GFP and tubulin proteins in Normal and ALS4 patient cells overexpressing GFP and ZPR1-GFP. FIG. 9F shows quantitation of SETX levels in Normal and ALS4 patient cells overexpressing GFP and ZPR1-GFP. FIG. 9G illustrates that IP of R-loops from Normal and ALS4 cells overexpressing GFP and ZPR1-GFP shows increase in in vivo binding of SETX with R-loops. FIG. 9H illustrates that quantitation and comparison of SETX levels in co-IP with R-loops show increase in SETX binding with R-loops (90.03±11.39%) compared to (34.24±4.30%, P=0.0101) in ALS4 expressing ZPR1-GFP and GFP, respectively.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Neurodegenerative disorders present numerous debilitating symptoms. For instance, amyotrophic lateral sclerosis 4 (ALS4) weakens muscles and impacts physical function due to the breakdown of nerve cells. Similarly, ataxia with oculomotor apraxia type 2 (AOA2) decreases a person's ability to move or feel sensation in certain parts of the body. Additional symptoms of AOA2 include movement disorders and mild cognitive impairment. Likewise, spinal muscular atrophy (SMA) and autosomal dominant SMA (ADSMA) present muscle weakness, decreased muscle tone, limited mobility, and breathing problems.

Furthermore, many neurodegenerative diseases and disorders (including ALS4, AOA2, SMA and ADSMA) remain incurable. Additionally, there are currently limited options for treating many neurodegenerative disorders.

As such, a need exists for improved therapeutics for treating or preventing neurodegenerative diseases and disorders. Numerous embodiments of the present disclosure address the aforementioned needs.

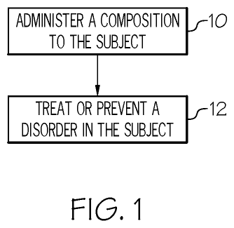

In some embodiments, the present disclosure pertains to methods of treating or preventing a disorder in a subject. In some embodiments illustrated in FIG. 1, the methods of the present disclosure include administering to the subject a composition (step 10). In some embodiments, the composition treats or prevents the disorder in the subject (step 12). Additional embodiments of the present disclosure pertain to compositions that are suitable for use in treating or preventing disorders.

As set forth in more detail herein, the methods and compositions of the present disclosure can have numerous embodiments. For instance, the compositions of the present disclosure can include various active components. Furthermore, various methods may be utilized to administer the compositions of the present disclosure to various subjects in order to treat and/or prevent various disorders in such subjects through various mechanisms.

Compositions

Generally, the compositions of the present disclosure include one or more active components. In some embodiments, the one or more active components include at least one of: (1) zinc finger protein ZPR1 (ZPR1), an analog thereof, a homolog thereof, a derivative thereof, or combinations thereof; (2) enhancers of expression of ZPR1, an analog thereof, a homolog thereof, or combinations thereof; (3) nucleotides encoding ZPR1, an analog thereof, a homolog thereof, a derivative thereof, or combinations thereof; or (4) combinations thereof.

ZPR1

In some embodiments, the one or more active components of the compositions of the present disclosure include ZPR1, an analog thereof, a homolog thereof, a derivative thereof, or combinations thereof. In some embodiments, the one or more active components of the compositions of the present disclosure include ZPR1. In some embodiments, ZPR1 is represented by a peptide sequence that includes SEQ ID NO: 1.

In some embodiments, ZPR1 includes a peptide sequence that shares at least 65% sequence homology to SEQ ID NO: 1. In some embodiments, ZPR1 includes a peptide sequence that shares at least 70% sequence homology to SEQ ID NO: 1. In some embodiments, ZPR1 includes a peptide sequence that shares at least 75% sequence homology to SEQ ID NO: 1. In some embodiments, ZPR1 includes a peptide sequence that shares at least 80% sequence homology to SEQ ID NO: 1. In some embodiments, ZPR1 includes a peptide sequence that shares at least 85% sequence homology to SEQ ID NO: 1. In some embodiments, ZPR1 includes a peptide sequence that shares at least 90% sequence homology to SEQ ID NO: 1. In some embodiments, ZPR1 includes a peptide sequence that shares at least 95% sequence homology to SEQ ID NO: 1.

ZPR1 Derivatives

In some embodiments, the one or more active components of the compositions of the present disclosure include a derivative of ZPR1. In some embodiments, the derivative of ZPR1 includes a recombinant ZPR1 fused to a green fluorescent protein (ZPR1-GFP).

ZPR1 Analogs

In some embodiments, the one or more active components of the compositions of the present disclosure include an analog of ZPR1. In some embodiments, the analog is at least 65% identical in peptide sequence to ZPR1. In some embodiments, the analog is at least 70% identical in peptide sequence to ZPR1. In some embodiments, the analog is at least 75% identical in peptide sequence to ZPR1. In some embodiments, the analog is at least 80% identical in peptide sequence to ZPR1. In some embodiments, the analog is at least 85% identical in peptide sequence to ZPR1. In some embodiments, the analog is at least 90% identical in peptide sequence to ZPR1. In some embodiments, the analog is at least 95% identical in peptide sequence to ZPR1.

ZPR1 Homologs

In some embodiments, the one or more active components of the compositions of the present disclosure include a homolog of ZPR1. In some embodiments, the homolog is at least 65% identical in peptide sequence to ZPR1. In some embodiments, the homolog is at least 70% identical in peptide sequence to ZPR1. In some embodiments, the homolog is at least 75% identical in peptide sequence to ZPR1. In some embodiments, the homolog is at least 80% identical in peptide sequence to ZPR1. In some embodiments, the homolog is at least 85% identical in peptide sequence to ZPR1. In some embodiments, the homolog is at least 90% identical in peptide sequence to ZPR1. In some embodiments, the homolog is at least 95% identical in peptide sequence to ZPR1.

Enhancers of ZPR1 Expression

In some embodiments, the one or more active components of the compositions of the present disclosure include an enhancer of ZPR1 expression. In some embodiments, the enhancer of ZPR1 expression is a compound that enhances the expression of ZPR1. In some embodiments, the enhancer of ZPR1 expression is a compound that selectively enhances the expression of ZPR1.

In some embodiments, the enhancer of ZPR1 expression is a compound or a combination of compounds that selectively enhances the expression of ZPR1. In some embodiments, the enhancer of ZPR1 expression is a gene that enhances the expression of ZPR1. In some embodiments, the enhancer of ZPR1 expression is a protein that enhances the expression of ZPR1.

ZPR1 Nucleotides

In some embodiments, the one or more active components of the compositions of the present disclosure include a nucleotide sequence encoding ZPR1, a homolog thereof, a derivative thereof, an analog thereof, or combinations thereof. In some embodiments, the nucleotide sequence is capable of expressing ZPR1 after administration to a subject. In some embodiments, the nucleotide sequence is in the form of DNA. In some embodiments, the nucleotide sequence is in the form of mRNA.

In some embodiments, the ZPR1 nucleotide sequence includes SEQ ID NO: 2. In some embodiments, the ZPR1 nucleotide sequence shares at least 65% sequence homology to SEQ ID NO: 2. In some embodiments, the ZPR1 nucleotide sequence shares at least 70% sequence homology to SEQ ID NO: 2. In some embodiments, the ZPR1 nucleotide sequence shares at least 75% sequence homology to SEQ ID NO: 2. In some embodiments, the ZPR1 nucleotide sequence shares at least 80% sequence homology to SEQ ID NO: 2. In some embodiments, the ZPR1 nucleotide sequence shares at least 85% sequence homology to SEQ ID NO: 2. In some embodiments, the ZPR1 nucleotide sequence shares at least 90% sequence homology to SEQ ID NO: 2. In some embodiments, the ZPR1 nucleotide sequence shares at least 95% sequence homology to SEQ ID NO: 2.

In some embodiments, the ZPR1 nucleotide sequence includes a mRNA transcript of SEQ ID NO: 2. In some embodiments, the ZPR1 nucleotide sequence includes a mRNA transcript of a nucleotide sequence that shares at least 65% sequence homology to SEQ ID NO: 2. In some embodiments, the ZPR1 nucleotide sequence includes a mRNA transcript of a nucleotide sequence that shares at least 70% sequence homology to SEQ ID NO: 2. In some embodiments, the ZPR1 nucleotide sequence includes a mRNA transcript of a nucleotide sequence that shares at least 75% sequence homology to SEQ ID NO: 2. In some embodiments, the ZPR1 nucleotide sequence includes a mRNA transcript of a nucleotide sequence that shares at least 85% sequence homology to SEQ ID NO: 2. In some embodiments, the ZPR1 nucleotide sequence includes a mRNA transcript of a nucleotide sequence that shares at least 90% sequence homology to SEQ ID NO: 2. In some embodiments, the ZPR1 nucleotide sequence includes a mRNA transcript of a nucleotide sequence that shares at least 95% sequence homology to SEQ ID NO: 2.

ZPR1 Derivatives

In some embodiments, the one or more active components of the compositions of the present disclosure include a derivative of a nucleotide sequence encoding ZPR1. In some embodiments, the derivative of ZPR1 includes a nucleotide sequence encoding ZPR1 fused to a nucleotide sequence encoding a green fluorescent protein (ZPR1-GFP).

In some embodiments, the nucleotide sequence is in an expression vector. In some embodiments, the expression vector expresses ZPR1 from the nucleotide sequence. In some embodiments, the expression vector includes, without limitation, a plasmid, a viral expression vector, or combinations thereof. In some embodiments, the expression vector includes a viral expression vector. In some embodiments, the viral expression vector includes an adenovirus expression vector.

Additional Components

The compositions of the present disclosure can include various additional components. For instance, in some embodiments, the composition can include at least one excipient agent. In some embodiments, the at least one excipient agent can include, without limitation, anti-adherents, binders, coatings, colors, disintegrants, flavors, glidants, lubricants, preservatives, sorbents, sweeteners, vehicles, or combinations thereof.

In some embodiments, the compositions of the present may also include a delivery vehicle. In some embodiments, the delivery vehicle includes particles. In some embodiments, the particles include, without limitation, nanoparticles, liposomes, or combinations thereof. In some embodiments, the delivery vehicle includes liposomes. In some embodiments, the one or more active components are encapsulated in the particles.

The compositions of the present disclosure may be suitable for various applications. For instance, in some embodiments, the compositions of the present disclosure may be suitable for use in treating or preventing a disorder in a subject. In some embodiments, the compositions of the present disclosure may be suitable for use in treating or preventing a disorder in a subject by administering the compositions of the present disclosure to the subject in accordance with the methods of the present disclosure.

Method for Treating and/or Preventing Disorders

Further embodiments of the present disclosure pertain to methods of treating or preventing a disorder in a subject by administering a composition of the present disclosure to the subject. As set forth in more detail herein, the methods of the present disclosure may utilize various modes of administration to administer the compositions of the present disclosure to various subjects in order to treat or prevent various disorders.

Administration of Compositions

Various methods may be utilized to administer the compositions the present disclosure to a subject. For instance, in some embodiments, the administering can include, without limitation, intravenous administration, subcutaneous administration, transdermal administration, topical administration, intraarterial administration, intrathecal administration, intracranial administration, intraperitoneal administration, intraspinal administration, intranasal administration, intraocular administration, oral administration, or combinations thereof.

Treatment or Prevention of Disorders

The methods of the present disclosure can be utilized to treat or prevent various types of disorders. For instance, in some embodiments, the disorder includes a disease or disorder caused by at least one mutation in the Senataxin (SETX) gene, a downregulation of SETX protein levels, or combinations thereof. In some embodiments, the disorder includes a disease or disorder caused by at least one mutation in the Senataxin (SETX) gene. In some embodiments, the disorder is a neurodegenerative disease or disorder. In some embodiments, the neurodegenerative disease or disorder includes, without limitation, amyotrophic lateral sclerosis-4 (ALS4), ataxia with oculomotor apraxia type 2 (AOA2), spinal muscular atrophy (SMA), autosomal dominant SMA (ADSMA), or combinations thereof. In some embodiments, the disorder includes ALS4. In some embodiments, the disorder includes AOA2. In some embodiments, the disorder includes SMA. In some embodiments, the disorder includes ADSMA. In some embodiments, the disorder includes SMA and ADSMA.

Without being bound by theory, the compositions and methods of the present disclosure can treat or prevent disorders through various molecular mechanisms. For instance, in some embodiments, an administered ZPR1 or an expressed ZPR1 from the active components of the compositions of the present disclosure treat or prevent the disorder by binding to co-transcriptional RNA-DNA hybrids (R-loops), recruiting Senataxin (SETX) onto the R-loops, and regulating the prevalence of the R-loops. In some embodiments, the regulating includes decreasing R-loop accumulation, such as by at least one-fold, at least two-fold, at least three-fold, or at least four-fold. In some embodiments, the regulating includes increasing R-loop accumulation, such as by at least one-fold, at least two-fold, at least three-fold, or at least four-fold. In some embodiments, the regulating includes modulating R-loop levels by increasing or decreasing R-loop levels to rescue optimal or physiological levels.

In some embodiments where the disorder to be treated or prevented is spinal muscular atrophy (SMA) and/or autosomal dominant SMA (ADSMA), the administered or expressed ZPR1 treats or prevents the SMA by decreasing R-loop accumulation. In some embodiments where the disorder to be treated or prevented is amyotrophic lateral sclerosis 4 (ALS4), the administered or expressed ZPR1 treats or prevents the SMA by increasing R-loop accumulation.

Without being bound by theory, the compositions and methods of the present disclosure can treat or prevent disorders through various molecular mechanisms. For instance, in some embodiments, the administered or expressed ZPR1 treats or prevents a disorder by reversing axonal defects of neurons. In some embodiments, the axonal defects include, without limitation, retraction, bending, folding, ballooning and combinations thereof.

Subjects

The compositions and methods of the present disclosure can be utilized to treat or prevent disorders in various subjects. For instance, in some embodiments, the subject is a mammal. In some embodiments, the subject is a human being.

In some embodiments, the subject is suffering from a disorder to be treated or prevented. In some embodiments, the subject is vulnerable to a disorder to be treated or prevented.

In some embodiments, the methods and compositions of the present disclosure may be utilized to treat a disorder in a subject. In some embodiments, the methods and compositions of the present disclosure may be utilized to prevent a disorder in a subject. In some embodiments, the methods and compositions of the present disclosure may be utilized to treat and prevent a disorder in a subject.

### Additional Embodiments

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. Mutation in Senataxin Alters the Mechanism of R-Loop Resolution in Amyotrophic Lateral Sclerosis 4

Mutation in the Senataxin (SETX) gene causes an autosomal dominant neuromuscular disorder amyotrophic lateral sclerosis 4 (ALS4), which is characterized by degeneration of motor neurons, muscle weakness and atrophy. SETX is an RNA-DNA helicase that mediates resolution of co-transcriptional RNA-DNA hybrids (R-loops). The process of R-loop resolution is essential for the normal functioning of cells, including neurons. The molecular basis of ALS4 pathogenesis and the mechanism of R-loop resolution are unclear. Applicant report in this Example that the zinc finger protein ZPR1 binds to RNA-DNA hybrids, recruits SETX onto R-loops and is critical for R-loop resolution. ZPR1 deficiency disrupts the integrity of R-loop resolution complexes (RLRC) containing SETX and causes increased R-loop accumulation throughout gene transcription. Applicant uncovers that SETX is a downstream target of ZPR1 and that overexpression of ZPR1 can rescue RLRC assembly in SETX-deficient cells but not vice versa.

To uncover the mechanism of R-loop resolution, Applicant examined the function of SETX-ZPR1 complexes using two genetic motor neuron disease models with altered R-loop resolution. Notably, chronic low levels of SETX-ZPR1 complexes onto R-loops result in a decrease of R-loop resolution activity causing an increase in R-loop levels in spinal muscular atrophy (SMA). ZPR1 overexpression increases recruitment of SETX onto R-loops, decreases R-loops and rescues the SMA phenotype in motor neurons and patient cells.

Interaction of SETX with ZPR1 is disrupted in ALS4 patients that have heterozygous SETX (L389S) mutation. ZPR1 fails to recruit the mutant SETX homodimer but recruits the heterodimer with partially disrupted interaction between SETX and ZPR1. Disruption of SETX-ZPR1 complexes causes increase in R-loop resolution activity leading to fewer R-loops in ALS4. Modulation of ZPR1 levels regulates R-loop accumulation and rescues the ALS4 phenotype in patient cells. These findings originate a new concept, “opposite alterations in a cell biological activity (R-loop resolution) result in similar pathogenesis (neurodegeneration) in different genetic motor neuron disorders”. Applicant proposes that ZPR1 collaborates with SETX and may function as a molecular brake to regulate SETX-dependent R-loop resolution activity critical for the normal functioning of motor neurons.

### Example 1.1. Background

Amyotrophic lateral sclerosis 4 (ALS4) is an autosomal dominant neuromuscular disorder caused by mutation in the Senataxin (SETX) gene. ALS4 is classified as a juvenile form of ALS and characterized by chronic degeneration of upper and lower motor neurons, distal muscle weakness and atrophy. SETX is an RNA-DNA helicase involved in the resolution of RNA-DNA hybrids (R-loops) formed during transcription.

R-loops consist of three nucleic acids strands, nascent RNA hybridized to the transcribing DNA strand (RNA-DNA hybrid) and a complementary DNA strand. R-loops play important roles in physiological processes, including immunoglobin (Ig) class switching, gene expression, DNA repair and genome instability. Defects in R-loop metabolism are associated with human diseases such as cancer and neurodegenerative disorders. Thus, precise regulation of R-loop resolution is critical for the normal functioning and survival of the cell.

The molecular mechanism of R-loop resolution is not well understood. Many factors have been identified that modulate R-loop levels or interact with R-loops, but their specific biochemical contribution to R-loop metabolism remains to be validated. One of the key factors is SETX, an ATP-dependent RNA/DNA helicase that unwinds RNA-DNA hybrids and contributes to R-loop resolution. Other critical factors that modulate RNA-DNA resolution during RNA polymerase II (RNAPII)-dependent gene transcription include XRN2, a 5′-3′-exonuclease that promotes SETX-dependent resolution of R-loops at G-rich transcription pause sites. RNA helicase A or DHX9 increases R-loop formation in cells with splicing defects and enhances transcription termination by suppressing R-loop accumulation. SETX forms complexes with RNAPII and survival motor neuron protein (SMN) and these protein complexes are involved in mRNA biogenesis that includes transcription, splicing and R-loop resolution. SMN directly interacts with RNAPII, and the disruption of RNAPII and SMN interactions by mutations in RNAPII causes defects in transcription termination. Mutation of the SMN1 gene causes spinal muscular atrophy (SMA). Chronic SMN deficiency causes downregulation of SETX, resulting in R-loop accumulation and DNA damage leading to genomic instability and neurodegeneration in SMA.

ZPR1 is evolutionarily conserved and is essential for cell viability in yeast and mammals. ZPR1 interacts with SMN and is required for SMN translocation from the cytoplasm to the nucleus. ZPR1 also interacts with RNAPII and is part of SMN-RNAPII complexes. ZPR1 deficiency causes neurodegeneration and contributes to respiratory distress associated with SMA pathogenesis. ZPR1 is a protective modifier, it upregulates expression of SMN and rescues SMA in mice. However, the physiological function of ZPR1 is unknown.

### Example 1.2. Summary of Findings

In this Example, Applicant demonstrates that the zinc finger protein ZPR1 forms complexes with SETX and R-loops. In particular, Applicant shows in this Example that ZPR1 binds to RNA-DNA hybrids, recruits SETX onto R-loops and is critical for the resolution of RNA-DNA hybrids. To unravel the molecular mechanism of R-loop resolution, Applicant investigated the role of ZPR1-SETX complexes using two disease models with altered R-loop metabolism: SMA with increased R-loops and ALS4 with decreased R-loops.

In SMA, chronic low levels of ZPR1-SETX complexes impair the efficiency of R-loop resolution resulting in R-loop accumulation. In ALS4, interaction of SETX with ZPR1 is disrupted in patients that have heterozygous SETX (L389S) mutation. ZPR1 fails to recruit mutant SETX homodimer but recruits heterodimer with partially disrupted interaction between SETX and ZPR1. Disruption of ZPR1-SETX complexes results in ZPR1-dependent gain in R-loop resolution activity leading to fewer R-loops in ALS4. Modulation of ZPR1 levels regulates SETX abundance, assembly and R-loop resolution activity of RLRC, and rescues pathogenic R-loop phenotype in ALS4 patient cells.

These data suggest that ZPR1 tethers to RNA-DNA hybrids, recruits SETX onto R-loops and may function as a molecular brake to regulate RLRC activity. Together, these findings originate a novel concept, opposite alterations in R-loop resolution activity result in similar pathogenesis, motor neuron degeneration, in different genetic motor neuron disorders, SMA and ALS4. Applicant proposes ZPR1 as a potential therapeutic target for manipulating R-loop levels under different disease conditions.

### Example 1.3. ZPR1 Forms Endogenous Complexes with SETX

ZPR1 is an evolutionarily conserved and ubiquitously expressed protein in eukaryotes and is essential for cell viability. However, the biochemical and physiological functions of ZPR1 are unknown. Here, Applicant began by investigating the interaction of ZPR1 with other proteins. Applicant previously showed that several proteins co-immunoprecipitate with ZPR1 from ³⁵S-methionine-labelled cell lysates.

Three ZPR1 interacting proteins were identified, the epidermal growth factor receptor (EGFR), eukaryotic translation elongation factor-1A (eEF1A) and SMN. A few other co-immunoprecipitating proteins, including a prominent protein band at the molecular weight (MW) ˜300 kDa, remain to be identified.

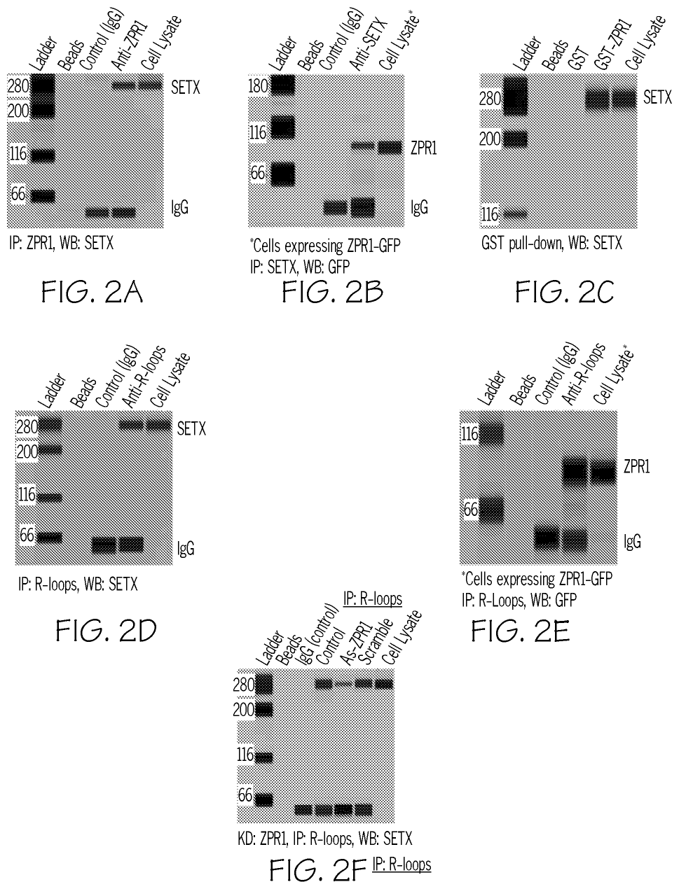

ZPR1 interacts and colocalizes with SMN, which interacts and colocalizes with SETX (˜300 kDa) in sub-nuclear bodies, raising the possibility that the 300 kDa MW protein might be SETX. To test this possibility, Applicant examined the interaction of endogenous ZPR1 with SETX by immunoprecipitation (IP) followed by automated western blot (WB) analysis. Applicant found that SETX co-immunoprecipitates with ZPR1 from HeLa cell lysates (FIG. 2A). Conversely, IP with SETX antibody shows that ZPR1 co-immunoprecipitates with SETX (FIG. 2B). For the SETX IP, Applicant used COS7 cells expressing ZPR1-GFP because ZPR1 MW is ˜52-54 kDa and migrates with heavy chain IgG, making it difficult to detect in WB analysis, whereas ZPR1-GFP MW is ˜80 kDa, runs above the IgG band and allows its unequivocal detection. Further, GST pulldown assay using purified recombinant GST-ZPR1 protein shows that ZPR1 can efficiently pulldown SETX from cell lysates (FIG. 2C). These data suggest that ZPR1 forms complexes with SETX in vivo.

### Example 1.4. ZPR1 Forms Endogenous Complexes with R-Loops

SETX binds to RNA-DNA hybrids and is an RNA/DNA helicase. ZPR1 contains two zinc fingers that may have affinity for binding to nucleic acids. To test whether ZPR1 binds to RNA-DNA hybrids, Applicant examined the binding of ZPR1 with labelled DNA and RNA-DNA hybrids using electrophoretic-mobility shift assay in vitro and found that this was indeed the case. Applicant also tested ZPR1 affinity for single-stranded (ssRNA) and double-stranded (dsRNA). These in vitro data show that ZPR1 has low binding affinity for ssRNA and did not bind to dsRNA.

To test the interaction of ZPR1 with R-loops in vivo, Applicant used an antibody against RNA-DNA hybrids (S9.6) used for the detection and IP of R-loops. IP of R-loops followed by WB showed that SETX co-immunoprecipitates with R-loops (FIG. 2D). Notably, Applicant found that ZPR1 also co-immunoprecipitates with R-loops (FIG. 2E).

To test the specificity of ZPR1 interaction with SETX and R-loops, Applicant examined the effect of ribonucleases treatment on in vivo interactions. Applicant found that the treatment of cell lysates with DNase I, RNase A and RNase H before IP did not affect the binding of ZPR1 with SETX, suggesting that the binding of ZPR1 with SETX is independent of RNA and DNA. Notably, treatments with RNase T1 (digest ssRNA), RNase III (digest dsRNA) and RNase H (digest RNA-DNA hybrids) show that only RNase H abolishes the binding of ZPR1 and SETX with R-loops. These data suggest that ZPR1 and SETX specifically bind to RNA-DNA hybrids and demonstrate the specificity of S9.6 antibody for the IP of RNA-DNA hybrid (R-loop) complexes with ZPR1 and SETX. Together, these data suggest that ZPR1 and SETX interact and may form endogenous complexes with R-loops.

### Example 1.5. ZPR1 is Critical for SETX Binding with R-Loops

The interaction between ZPR1 and SETX and their association with R-loops suggest that they may collaborate to regulate R-loop resolution. To gain mechanistic insight into this process, Applicant examined the effect of ZPR1 knockdown on SETX binding with R-loops. ZPR1 knockdown in HeLa cells causes decrease in ZPR1 levels to ˜22% compared to control and scramble treated cells. IP of R-loops using S9.6 antibody shows that ZPR1-deficiency causes marked decrease in the binding of SETX with R-loops in vivo (FIG. 2F). Quantitation shows that SETX co-immunoprecipitation decreased to ˜25% in ZPR1-deficient cells. Notably, knockdown of SETX did not affect the binding of ZPR1 to R-loops.

To further test whether ZPR1 is critical for SETX binding to R-loops, Applicant examined the effect of SETX overexpression on the rescue of SETX binding with R-loops in ZPR1-deficient cells. The quantitative data show that SETX binding was decreased by ˜61% in ZPR1-deficient cells (As-ZPR1) and ˜65% in ZPR1-deficient cells with SETX overexpression (As-ZPR1+SETX-OE), suggesting that SETX overexpression did not rescue SETX binding with R-loops in ZPR1-deficient cells. These data suggest that ZPR1 is required for in vivo binding of SETX with R-loops and support the idea that ZPR1 may recruit SETX onto R-loops.

Together, these data suggest that ZPR1 recruits SETX onto R-loops and is critical for in vivo assembly of the core complex of proteins and nucleic acids, SETX-ZPR1-RNA-DNA hybrids. Applicant call this the “R-loop resolution complexes (RLRC)” to illustrate its critical role in resolving RNA-DNA hybrids formed during transcription.

### Example 1.6. ZPR1-Deficiency Causes Downregulation of SETX and Accumulation of R-Loops

To test whether ZPR1 contributes to the physiological function of SETX in R-loop resolution, Applicant investigated the effect of ZPR1 deficiency on SETX cellular distribution and R-loop accumulation. SETX colocalizes with SMN in nuclear gems. ZPR1 is required for SMN and p80 coilin accumulation in sub-nuclear bodies, including gems and Cajal bodies (CBs). Applicant found that ZPR1 colocalizes with SETX and is required for the accumulation of SETX in sub-nuclear bodies, including gems and CBs in HeLa and WI-38 cells. Control HeLa cells showed co-localization of SETX with ZPR1, SMN and coilin in sub-nuclear bodies.

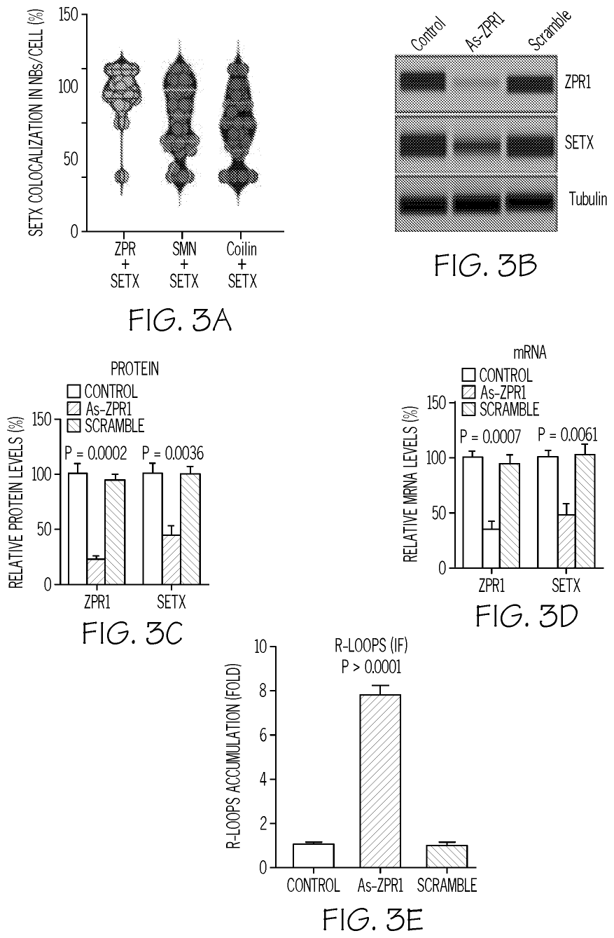

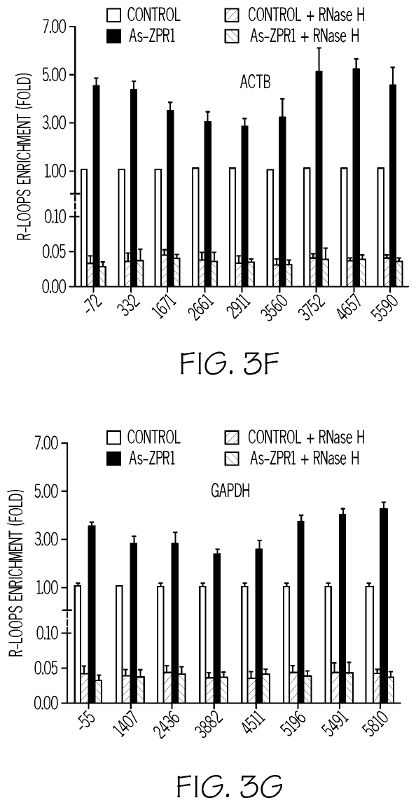

Quantification of SETX colocalization show the highest colocalization with ZPR1 (79.60±3.03%) compared to SMN (54.01±4.51%) (gems) and coilin (47.00±4.46%) (CBs) (FIG. 3A). Quantitative and IF analyses of SETX colocalization in WI-38 cells show similar trend and the highest SETX colocalization with ZPR1 (96.00±3.77%). These data show that the majority of SETX⁺ nuclear bodies colocalize with ZPR1 and suggest functional collaboration between ZPR1⁺ and SETX⁺ nuclear bodies. Notably, knockdown of ZPR1 (As-ZPR1) causes disruption of SETX⁺, SMN⁺ (gems) and coilin⁺ (CBs) and decreases the staining intensity of SETX compared to control and scramble treated cells. These data suggest that ZPR1 is required for SETX localization in gems and CBs, and indicate that ZPR1 deficiency may cause defects in SETX function.

SETX is an ATP-dependent helicase required for unwinding and resolution of RNA-DNA hybrids formed during transcription. To test whether disruption of SETX⁺ bodies upon ZPR1 knockdown correlates with altered R-loop resolution, Applicant examined the effect of ZPR1-deficiency on R-loops using an antibody against RNA-DNA hybrids (S9.6) that detects R-loops. Control cells show low levels of R-loops in the nucleus compared to cytoplasm, where they are associated with mitochondrial transcription. Notably, knockdown of ZPR1 (As-ZPR1) causes marked accumulation of R-loops in the nucleus. Staining of ZPR1-deficient cells with an antibody against γH2AX, a marker for DNA damage, shows accumulation of γH2AX foci in the nucleus, which indicates that ZPR1-deficiency may cause R-loop-mediated DNA damage.

To test the specificity of the S9.6 antibody for R-loop detection, Applicant treated control and transfected (As-ZPR1) cells with permeabilization buffer without or with RNase H that digests R-loops. Control and ZPR1-deficient cells (As-ZPR1) cells show that R-loops can be resolved by digestion of RNA using exogenous RNase H, establishing the specificity of R-loop detection and accumulation. Further, complementation of ZPR1-deficient cells (As-ZPR1) with mouse ZPR1-GFP causes decrease in R-loops and rescues DNA damage accumulation. Together, these results establish that ZPR1 deficiency negatively impacts on R-loop resolution.

The reduced staining of SETX in ZPR1-deficient cells indicates that ZPR1 may influence SETX levels. Quantitative analysis of immunoblots shows that knockdown of ZPR1 levels to ˜22% (P=0.0002) causes ˜56% (P=0.0036) decrease in SETX protein levels (FIGS. 3B and 3C) compared to control and scramble treated cells. Analysis of mRNA levels shows that ZPR1 knockdown to ˜34% (P=0.0007) causes downregulation of SETX mRNA expression to ˜47% (P=0.0061) compared to control and scramble (FIG. 3D). These data suggest that SETX may be a downstream target of ZPR1. Quantitation of nuclear R-loops revealed by immunostaining shows ZPR1-deficiency (FIG. 3E) causes marked increase (˜7.80-fold, P<0.0001) in R-loop accumulation.

To further investigate the role of ZPR1 in R-loop resolution, Applicant mapped the accumulation of R-loops during transcription of beta-actin (ACTB) and glyceraldehyde 3-phosphate dehydrogenase (GAPDH) genes using DNA-RNA hybrid immunoprecipitation (DRIP) with and without RNase H treatment followed by real-time qPCR. Analysis of ACTB gene transcripts shows that ZPR1 deficiency (As-ZPR1) causes ˜4-5-fold R-loop accumulation throughout the transcription (FIG. 3F). Similarly, GAPDH gene transcripts show that ZPR1 deficiency (As-ZPR1) causes ˜3-4-fold R-loop accumulation throughout the transcription (FIG. 3G). Together, these data suggest that ZPR1 is critical for R-loop resolution.

### Example 1.7. SETX-Deficiency Causes Disruption of ZPR1⁺ NBs, Gems and Cajal Bodies, and Accumulation of R-Loops

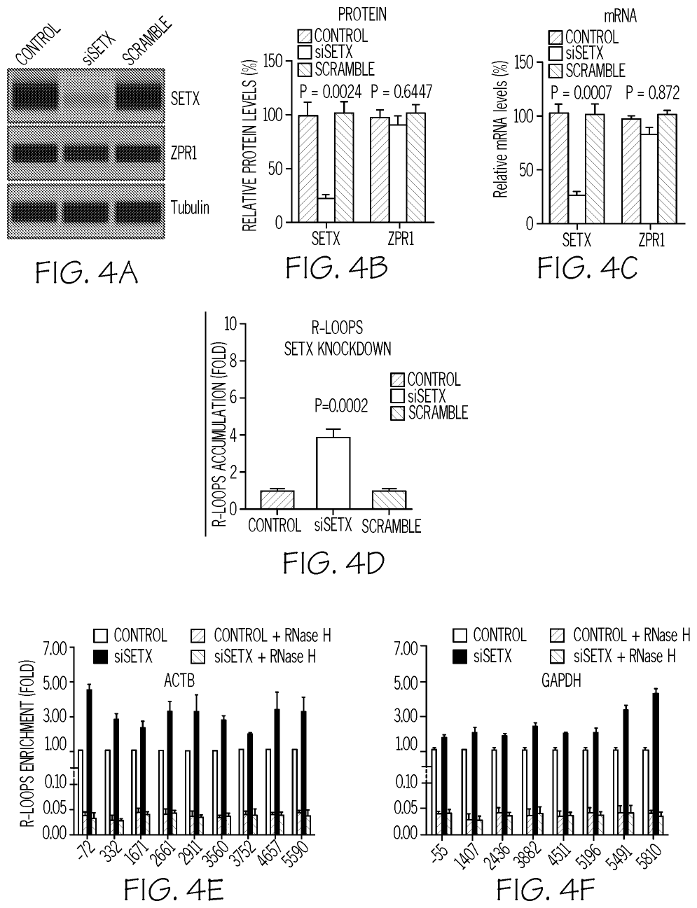

The disruption of SETX NBs and the downregulation of SETX in ZPR1-deficient cells suggest that ZPR1 may regulate SETX abundance and raise the question of whether SETX might also influence the integrity of NBs and ZPR1 levels. Knockdown of SETX (siSETX) causes disruption of ZPR1⁺, SMN⁺ (gems) and coilin⁺ (CBs) and results in mis-localization of ZPR1 in the nucleoplasm, fragmentation of gems and CBs in smaller nuclear foci compared to control and scramble treated cells. In contrast to ZPR1, knockdown of SETX (siSETX) and reduction of its levels to ˜22% (P=0.0024) did not significantly decrease ZPR1 levels, which remained ˜90% (P=0.6447) (FIGS. 4A and 4B). This result was supported by mRNA analysis that shows knockdown of SETX expression to ˜26% (P=0.0007) did not significantly alter ZPR1 mRNA expression ˜82% (P=0.0872) compared to control and scramble (FIG. 4C).

However, SETX knockdown causes disruption of ZPR1⁺ sub-nuclear bodies and redistributes ZPR1 in the nucleoplasm. Disruption of NBs and mis-localization of ZPR1 in SETX-deficient cells suggest that SETX-deficiency may influence ZPR1-dependent R-loop resolution. SETX-deficiency causes accumulation of R-loops and 53BP1 foci, a marker of DNA damage, in the nucleus, effects similar to the ones caused by ZPR1-deficiency. Quantitation of nuclear R-loops revealed by immunostaining shows SETX-deficiency causes ˜3.85-fold R-loop accumulation (FIG. 4D), which is lower than ZPR1-deficiency (˜7.80-fold) (FIG. 3E).

Analysis of R-loops during transcription of ACTB and GAPDH genes using DRIP with and without RNase H treatment followed by real-time qPCR shows that SETX-deficiency (siSETX) causes ˜2-3-fold (ACTB) and ˜1-3-fold (GAPDH) R-loop accumulation throughout the transcription (FIGS. 4E and 4F). Comparison of the effect of ZPR1 and SETX deficiencies shows that ZPR1-deficiency results in higher ˜4-fold accumulation of R-loops around the start of transcription (−72, 5′-UTR) and (−55, 5′-UTR) regions compared to SETX-deficiency in ACTB and GAPDH genes, respectively (FIGS. 3F and 3G and FIGS. 4E and 4F).

These data suggest ZPR1 may be critical for initiating R-loop resolution at the start of transcription. Together, these data demonstrate that SETX-dependent mis-localization of ZPR1 may be a cause of R-loop accumulation in SETX-deficient cells and indicate a functional contribution of SETX in ZPR1-dependent resolution of R-loops.

### Example 1.8. Chronic Low Levels of ZPR1 Impair Assembly of R-Loop Resolution Complexes (RLRC) in SMA

SMA is a motor neuron disorder caused by mutations in the SMN1 gene that result in low levels of SMN and neurodegeneration. Chronic SMN deficiency causes accumulation of pathogenic R-loops and DNA damage leading to genomic instability and neurodegeneration in SMA. However, the molecular mechanism of R-loop accumulation in SMA is unclear.

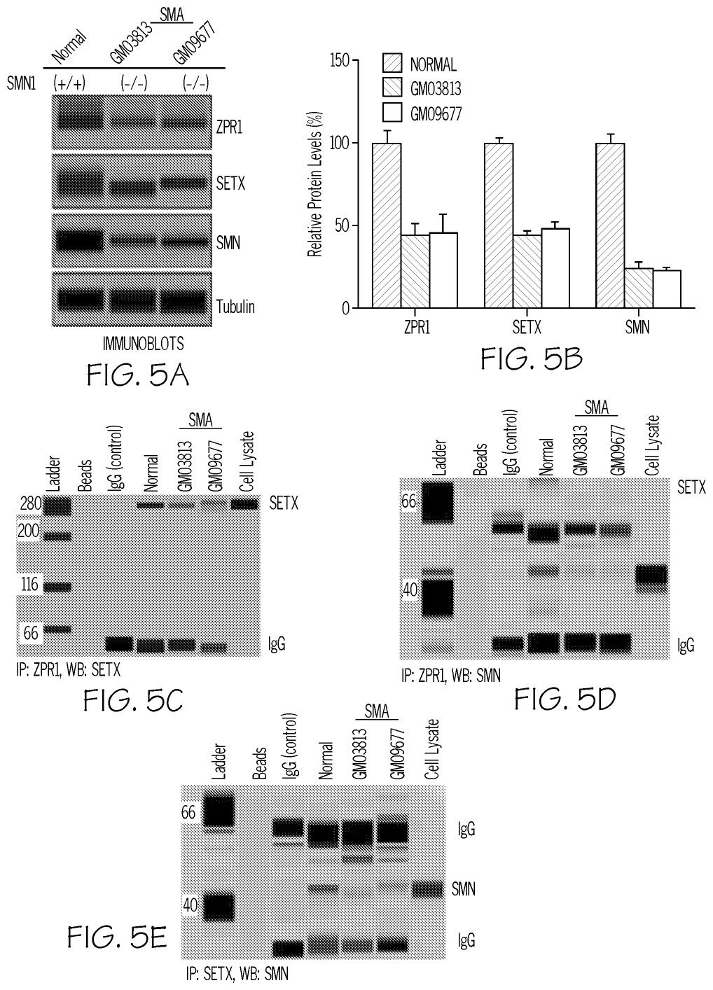

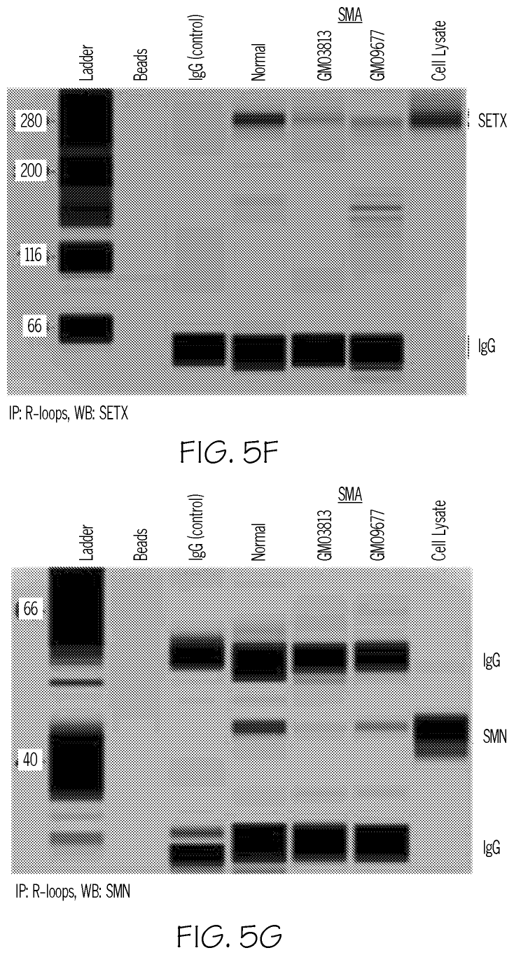

SMN forms in vivo complexes with SETX. Interestingly, ZPR1 is downregulated in SMA patients. It is possible that ZPR1 deficiency may contribute to R-loop accumulation associated with SMA pathogenesis. To test this possibility, Applicant examined in vivo binding of SETX and SMN with R-loops using fibroblasts derived from two SMA patients. Quantitative analysis shows low levels (˜45%) of ZPR1 in SMA patient fibroblasts compared to non-SMA (normal) fibroblasts (FIGS. 5A and 5B). SETX levels were also decreased ˜46% in SMA (FIGS. 5A and 5B).

These data suggest that chronic low levels of ZPR1 in SMA correlate with SETX downregulation, which is consistent with data in Example 1.6 and supports the idea that SETX may be a downstream target of ZPR1. Colocalization of ZPR1 with SETX, and colocalization of SMN with SETX in sub-nuclear foci is disrupted in SMA compared to normal cells, indicating that the disruption of sub-nuclear bodies and mislocalization of core components of RLRC may be a cause of R-loop accumulation. Analysis of cells stained with an antibody against R-loops and quantitation of nuclear R-loop IF intensity shows ˜2.5-fold higher R-loop accumulation in SMA patient cells compared to control cells.

To test whether R-loop accumulation might be because of defective assembly of RLRC core proteins onto R-loops, Applicant examined RLRC assembly in SMA. Analysis of ZPR1 endogenous complexes shows decrease in co-immunoprecipitation of SETX (FIG. 5C) and SMN (FIG. 5D) in SMA. Further, IPs of SETX and SMN show decrease in endogenous SETX-SMN complexes in SMA (FIG. 5E). These data thus reveal decreased levels of ZPR1-SETX, ZPR1-SMN and SETX-SMN complexes or interactions that are critical for R-loop resolution. IP of R-loops shows marked decrease in in vivo association of SETX (FIG. 5F) and SMN (FIG. 5G) with R-loops. Altogether, these results suggest that ZPR1 downregulation causes defects in the assembly of RLRC, which may lead to inefficient R-loop resolution and result in R-loop accumulation in SMA.

### Example 1.9. ZPR1 Rescues Defective RLRC Assembly and Prevents Pathogenic R-Loop Accumulation in SMA

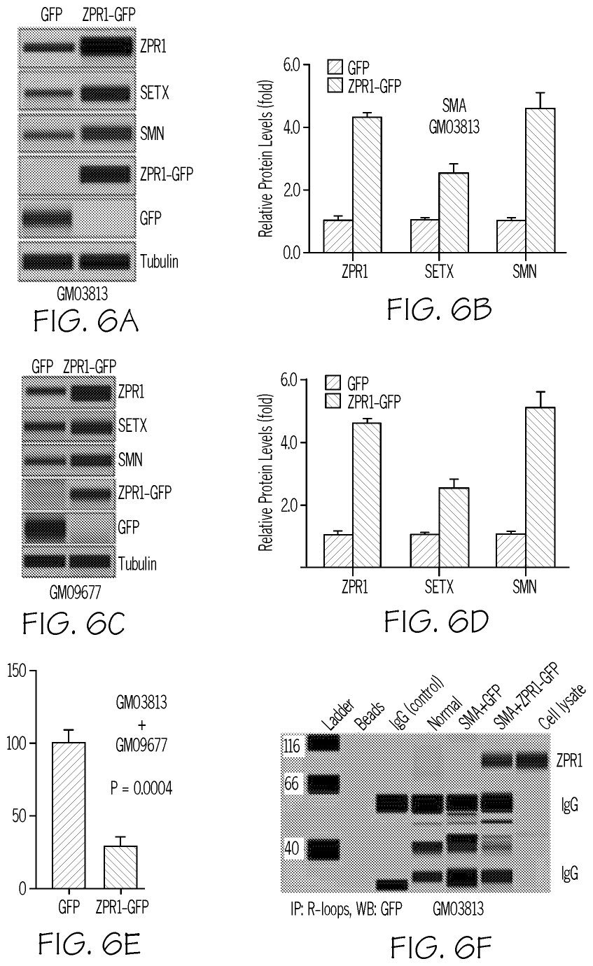

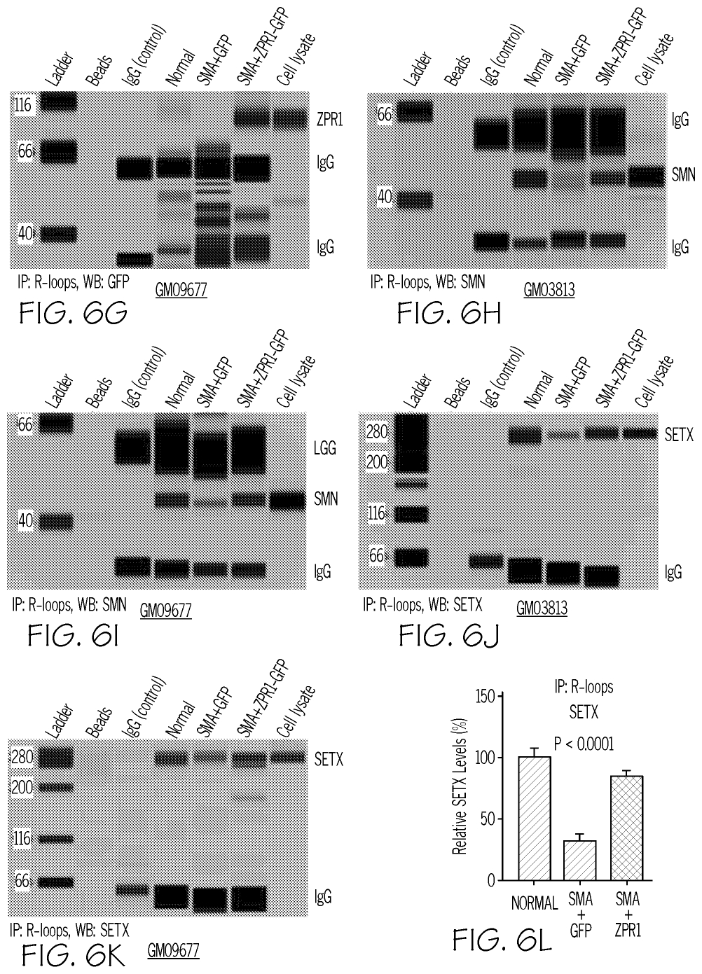

To test the hypothesis that ZPR1 is central to R-loop resolution, Applicant performed the rescue experiment with ZPR1 overexpression in SMA patient cells. Control cells expressing GFP did not show any change in the levels or cellular localization of SETX and SMN (GFP panels). Notably, ZPR1 overexpression increased SETX and SMN accumulation in the nucleus of GM03813 and GM09677 cells expressing ZPR1-GFP (arrows) compared to cells without expression (non-transfected, indicated by asterisks). Increase in ZPR1 (˜4-fold) expression increases the levels of SETX (˜2.5-fold) and SMN (˜4.5) in GM03813 cells (FIGS. 6A and 6B) and in GM09677 cells, SETX (˜2.5-fold) and SMN (˜5.0-fold) (FIGS. 6C and 6D).

These data suggest that modulation in ZPR1 levels alters the expression of SETX and SMN and support the idea that these critical proteins are downstream targets of ZPR1. Next, Applicant tested whether increase in ZPR1, SETX and SMN levels would reduce R-loop accumulation. Applicant found that both SMA cell lines, GM03813 and GM09677, overexpressing ZPR1-GFP show decrease in R-loop accumulation compared to non-transfected controls. ZPR1 overexpression causes ˜71% reduction in the accumulation of R-loops in SMA patient cells (FIG. 6E).

The decrease in R-loop levels in ZPR1 overexpressing SMA patient cells suggests that ZPR1 might rescue the assembly of RLRC and improve the efficiency of R-loop resolution. Analysis of R-loop IPs shows that ZPR1-GFP co-immunoprecipitates in patient cells GM03813 (FIG. 6F) and GM09677 (FIG. 6G).

Applicant has shown previously that ZPR1-GFP retains its biological activity and rescues viability of ZPR1-null cells. ZPR1 overexpression increases SMN co-immunoprecipitation with R-loops in SMA patient cell lines GM03813 (FIG. 6H) and GM09677 (FIG. 6I). Notably, ZPR1 also increases in vivo association of SETX with R-loops in ZPR1 complemented compared to control SMA patient cells, GM03813 (FIG. 6J) and GM09677 (FIG. 6K). IP of R-loops from SMA cells (SMA+GFP) shows low levels (˜32%) of SETX co-IP compared to normal cells. R-loop IPs of ZPR1 complemented cells (SMA+ZPR1-GFP) show higher levels (˜85%) of SETX co-IP, ˜2.5-fold increase, compared to control (SMA+GFP) cells (FIG. 6L). Together, these data show that ZPR1 overexpression recruits more SETX and SMN, and improves the assembly of RLRC, which enhances R-loop resolution and rescues the pathogenic R-loop phenotype in SMA patient cells. These data support Applicant's hypothesis that ZPR1 is critical for recruiting SETX and may be central to the process of R-loop resolution.

### Example 1.10. ZPR1 Overexpression In Vivo Rescues DNA Damage Associated with R-Loop Accumulation and Prevents Degeneration of Motor Neurons in SMA

SMA is a neurodegenerative disorder characterized by degeneration of spinal cord motor neurons. To test another important aspect of Applicant's hypothesis as to whether ZPR1 rescues molecular defects in vivo and specifically in SMA spinal cord motor neurons, Applicant used primary cultured spinal cord neurons derived from 7-day-old normal, SMA and transgenic mice Z-SMA (SMA mice overexpress Flag-Zpr1 under the control of mouse Rosa26 promoter) mice. Combined deficiency of SETX and DNA-activated protein kinase catalytic subunit (DNA-PKcs), critical for non-homologous end joining (NHEJ)-mediated DNA repair in neurons, causes R-loop accumulation and DNA damage, and inefficient DNA damage repair leading to genomic instability and motor neuron degeneration in SMA.

To test whether ZPR1 overexpression will restore SETX and DNA-PKcs levels and reduce DNA damage in motor neurons, Applicant examined and compared primary spinal cord neurons cultured from Normal, SMA and Z-SMA mice using IF and immunoblot analyses. Applicant established a method for culture of primary spinal cord neurons from postnatal mice, which stain positive for known motor neuron markers, including ChAT and Hlxb9 (Hb9), suggesting that cultured spinal cord neurons may retain characteristics of motor neurons.

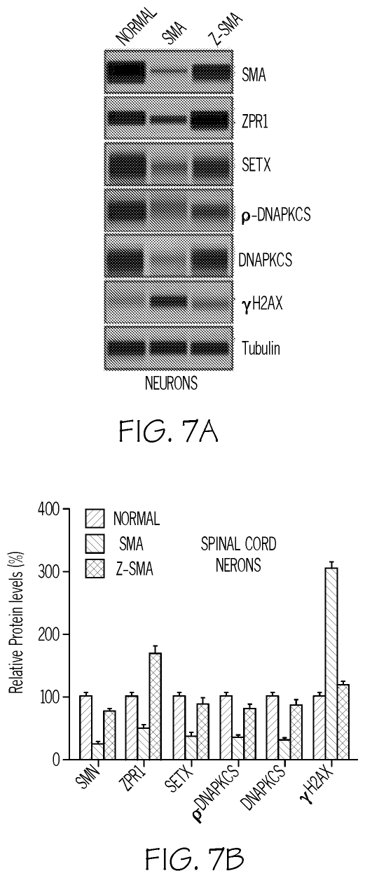

Comparison of morphology of cultured primary neurons stained with neuron-specific (3-tubulin-III showed that neurons from Z-SMA mice were healthy and were rescued of axonal defects such as retraction, bending, folding of axons (shown with arrowheads) compared to neurons from SMA mice. Staining of neurons for ZPR1 shows increased ZPR1 levels in neurons from Z-SMA mice, which is supported by immunoblot quantitative analysis (FIGS. 7A and 7B). Notably, increase in ZPR1 levels results in increased staining and levels of SMN, SETX and p-DNA-PKcs (FIGS. 7A and 7B). The increase in SETX levels (˜2.3-fold) suggests that there might be a decrease in the accumulation of R-loops, a concept supported by quantitative analysis of nuclear R-loops, which shows marked decrease (˜4.7-fold) in R-loop levels in Z-SMA compared to SMA neurons. Increase in total DNA-PKcs (˜2.69-fold) and activated p-DNA-PKcs (˜2.28-fold) levels suggest an improvement in the efficiency of DNA repair and reduction in DNA damage, further supported by IF and IB analyses of 7H2AX, a DNA damage marker, which show ˜2.6-fold decrease in 7H2AX levels (FIGS. 7A and 7B). These data suggest that ZPR1 overexpression restores levels of SETX and DNA-PKcs, critical for R-loop resolution and DNA damage repair respectively, and rescues R-loop-mediated DNA damage overall preventing degeneration of SMA neurons.

### Example 1.11. Interaction of SETX with ZPR1 is Disrupted in ALS4 Patients

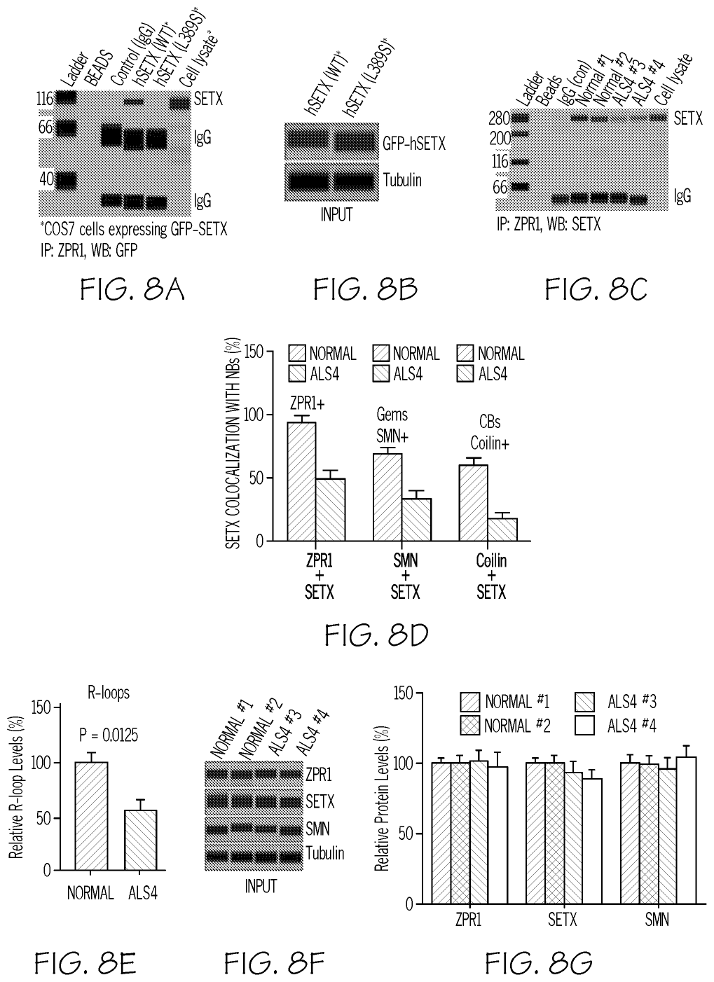

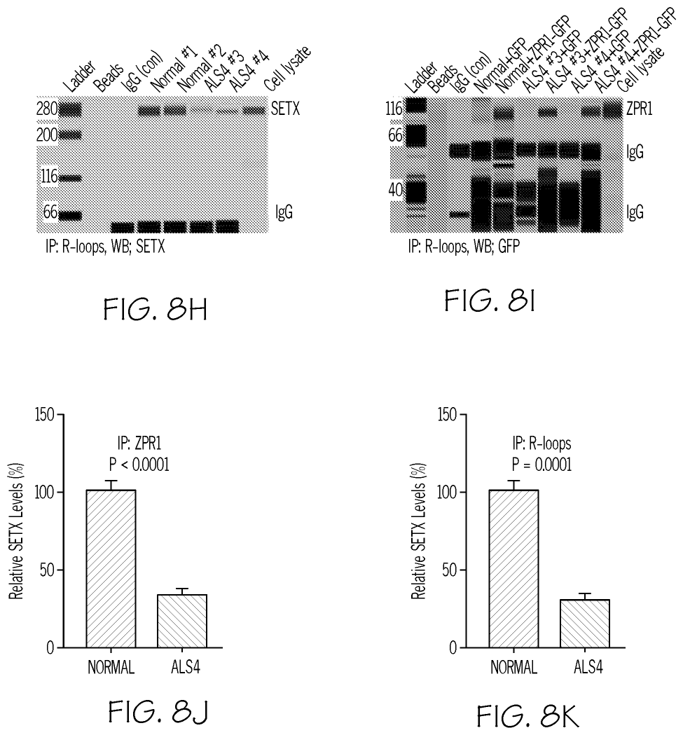

To further test Applicant's hypothesis that ZPR1 and SETX collaborate to regulate R-loop metabolism, Applicant thought that disruption of ZPR1 interaction with SETX might provide additional insight into the function of ZPR1-SETX complexes. The NH₂-terminal of SETX is involved in protein-protein interaction. Applicant anticipated that mutations reported in the NH₂-terminal of SETX might disrupt its interaction with ZPR1. Mutational analysis using recombinant SETX protein revealed that SETX mutation L389S, which causes ALS4, disrupts interaction of SETX with ZPR1 (FIGS. 8A and 8B). Based on these data, Applicant anticipated that the interaction between SETX and ZPR1 might be disrupted in ALS4.

To test the aforementioned hypothesis, Applicant examined interaction of SETX with ZPR1 using fibroblasts derived from ALS4 patients that have the SETX L389S mutation. Applicant found that SETX co-immunoprecipitation with ZPR1 was reduced in experiments with fibroblasts isolated from three different ALS4 patients compared to fibroblasts isolated from three normal subjects (FIG. 8C). These data indicate partial loss of SETX-ZPR1 complexes. This is likely because ALS4 is an autosomal dominant disease caused by heterozygous mutation, SETX^{+/L389S}, in the SETX gene. These data indicate that mutation in SETX disrupts ZPR1-SETX complexes in ALS4.

### Example 1.12. Mis-Localization of SETX and ZPR1 in ALS4 Patient Cells

The observation that SETX-ZPR1 complexes are disrupted in ALS4 suggests that the mutation in SETX might also affect cellular localization and alter levels of SETX and ZPR1. Applicant examined cellular distribution and levels of ZPR1 and SETX in ALS4 patient cells. Control (normal) cells show ZPR1 colocalizes with SETX. In contrast, ALS4 patient cells show disruption of co-localization of ZPR1 and SETX containing sub-nuclear bodies. In addition, the size of ZPR1 and SETX sub-nuclear bodies is reduced. SETX also colocalizes with SMN and coilin in control cells from normal subjects. Notably, SETX colocalization with SMN⁺ (gems) and coilin+ (CBs) is markedly reduced in ALS4 patient cells.

Quantification of SETX colocalization in normal cells (mean±SEM) shows higher colocalization with ZPR1 (94.33±5.36%) compared to SMN (69.67±5.28%) (gems) and Coilin (60.33±5.77%) (CBs) (FIG. 8D). Notably, SETX colocalization in ALS4 patient cells also show higher colocalization with ZPR1 (49.33±7.25%) compared to SMN (34.17±6.18%) and Coilin (18.00±4.23%) but the overall SETX colocalization is reduced by ˜48% (ZPR1), ˜51% (SMN) and ˜70% (coilin) in ALS4 compared to normal cells. Cellular mis-localization of ZPR1, SMN and coilin and disruption of SETX interaction with ZPR1 indicate the possibility of altered R-loop resolution in ALS4. IF and dot-blot quantifications show R-loop levels were reduced to (˜54%) and (˜42%), respectively, in ALS4 patient cells compared to control cells (FIG. 8E), which is consistent with recently published findings that hypothesized gain-of-function in R-loop resolution activity because of the mutation in SETX. Notably, comparison of ZPR1, SETX and SMN protein levels did not show any significant difference between control and ALS4 patient cells (FIGS. 8F and 8G). These findings suggest that disruption of SETX-ZPR1 complexes may be the cause of gain-of-function in R-loop resolution activity leading to fewer R-loops in ALS4.

### Example 1.13. Mutation in SETX Decreases In Vivo Association with R-Loops in ALS4

It is possible that the interaction of ZPR1 with SETX is critical for the recruitment of SETX onto R-loops. Therefore, the disruption of ZPR1 interaction with SETX in ALS4 may impair recruitment of mutant SETX (SETX*) onto R-loops. To test this, Applicant examined the effect of disruption of SETX-ZPR1 complexes on the binding of SETX with R-loops using ALS4 patient fibroblasts. Applicant found that SETX co-immunoprecipitation with R-loops was markedly reduced from ALS4 patient fibroblasts (SETX^{+/L389S}) compared to fibroblasts isolated from normal subjects (SETX^{+/+}) (FIG. 8H). To determine whether the disruption of ZPR1-SETX interaction also affects ZPR1 binding with R-loops, Applicant performed IP of R-loops followed by immunoblot analysis of ZPR1. Applicant found that in vivo association of ZPR1 with R-loops was unaffected in ALS4 patient cells (FIG. 8I). These data show that disruption of SETX interaction with ZPR1 results in decreased association of SETX with R-loops in vivo and support the idea that interaction of ZPR1 with SETX is critical for ZPR1 to recruit SETX onto R-loops.

Quantitative analysis of SETX co-immunoprecipitation with ZPR1 and R-loops shows a marked reduction in the amount of SETX immunoprecipitation; ˜34% with ZPR1 and ˜31% with R-loops (FIGS. 8J and 8K) in ALS4 compared to controls (Normal) fibroblasts. These data show unanticipated low levels of SETX co-immunoprecipitation, while SETX levels are unchanged in ALS4 compared to control fibroblasts (FIGS. 8F and 8G). However, theoretically 50% of the produced SETX protein would be mutant SETX* in ALS4 patients (SETX^{+/L389S}). In addition, SETX forms a homodimer and SETX mutation L389S does not affect its dimerization. These findings suggest that SETX may form three types of dimers, SETX-SETX, SETX-SETX* and SETX*-SETX*, with ˜33.3% contribution of each to total SETX pool in ALS4.

Notably, ZPR1 does not self-dimerize and does not form homodimers. Thus, the observation that mutation in SETX disrupts its interaction with ZPR1 suggests that SETX and ZPR1 may form two types of complexes with 1:1 and 1:0.5 stoichiometry, namely ZPR1-SETX-SETX-ZPR1 (normal) and ZPR1-SETX-SETX* (ALS4), respectively. It is possible that SETX heterodimers containing mutant SETX* (SETX-SETX*) may have reduced efficiency of recruitment by ZPR1 onto R-loops, which is supported by decreased SETX binding (˜31-34%) with ZPR1 and R-loops in ALS4. Applicant's data show that ZPR1 tethers to RNA-DNA hybrids, recruits SETX onto R-loops and may functions as a “molecular brake” to regulate SETX-dependent RLRC activity. Mutation in SETX disrupts its binding with ZPR1 that may cause partial impairment of the molecular brake resulting in higher activity of R-loop resolution (gain-of-function) leading to fewer R-loops in ALS4. Together, these data suggest a functional collaboration between SETX and ZPR1 in regulating R-loop resolution activity.

### Example 1.14. Modulation of ZPR1 Levels Regulates R-Loop Accumulation and Rescues Pathogenic R-Loop Phenotype in ALS4 Patient Cells

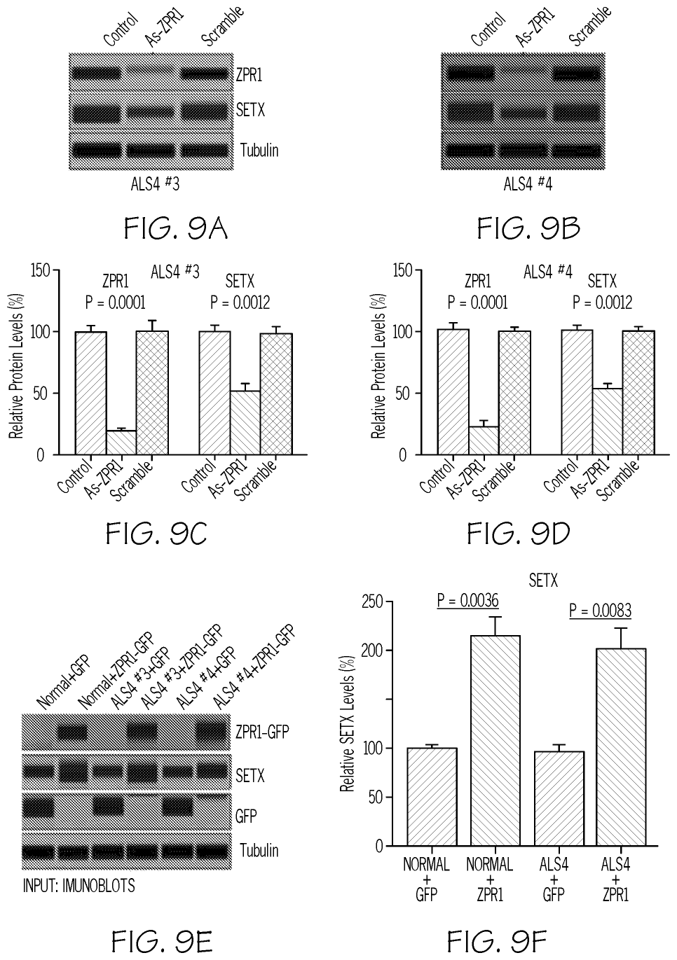

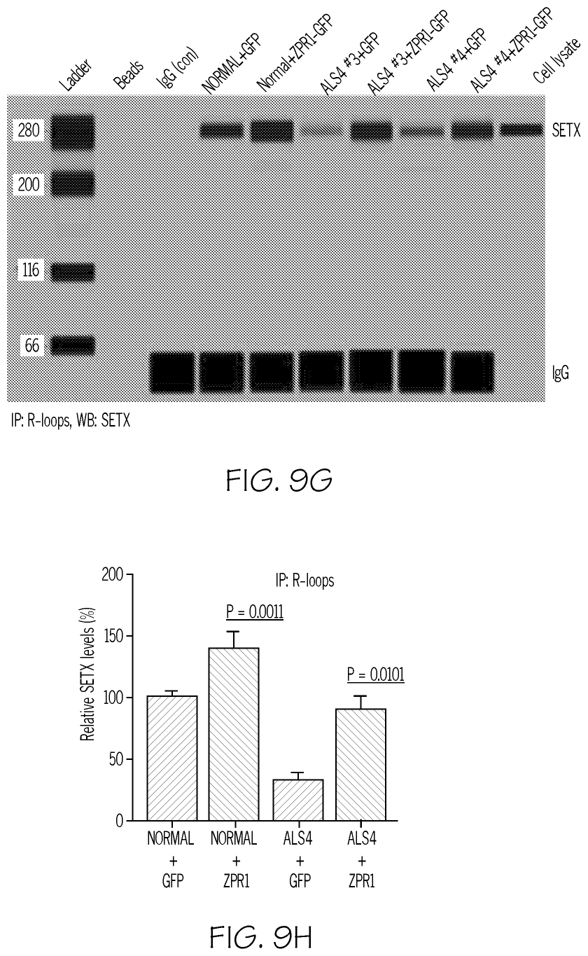

Comparison of data from SMA and ALS4 fibroblasts show similar decrease (˜65%) in SETX association with R-loops but contrasting levels of R-loop accumulation in SMA (high) and ALS4 (low). These data raise the question of whether ALS4 patient cells have normal biochemical potential to accumulate R-loops. To address this, Applicant examined the effect of ZPR1 knockdown on R-loop accumulation. Applicant found that ZPR1 knockdown causes R-loop accumulation in both ALS4 fibroblasts lines (As-ZPR1) compared control and scramble oligo treated ALS4 fibroblasts. Thus, ALS4 cells retain the biochemical potential and the molecular machinery for R-loop accumulation. Applicant also found that ZPR1-deficient ALS4 fibroblasts accumulated 53BP1 and γH2AX foci, markers for DNA damage and double strand breaks. Quantitative analysis of ZPR1 knockdown shows ˜50% downregulation of SETX in both ALS4 cell lines suggesting that downregulation of mutant SETX* may also contribute to R-loop accumulation in ZPR1-deficient cells (FIGS. 9A-H). Thus, R-loop accumulation in ALS4 cells triggers similar downstream molecular events, which compromise genomic integrity, with those observed and reported in control and SMA cells.

Further, Applicant examined the effect of ZPR1 overexpression on R-loop accumulation in ALS4 patient cells using adenoviral infection Ad-GFP (GFP) and Ad-ZPR1-GFP (ZPR1-GFP). Control experiment did not show any change in R-loop staining of cells expressing GFP (green, arrows) compared to cells that were not infected (asterisks) in normal and ALS4 fibroblasts. Notably, normal subject-derived fibroblasts expressing ZPR1-GFP showed marked decrease in R-loop staining compared to non-infected cells. These data suggest that ZPR1 has the potential to accelerate R-loop resolution under normal conditions.

Interestingly, overexpression of ZPR1 in ALS4 patient fibroblasts causes increase in accumulation of R-loops compared to non-infected cells, suggesting that ZPR1 can rescue cellular phenotype associated with ALS4 pathogenesis. To gain insight into the mechanism of rescue of pathogenic ALS4 phenotype, Applicant examined the effect of ZPR1 overexpression on levels of SETX and its in vivo association with R-loops. Applicant found ˜2.0-fold increase in SETX levels in normal and ALS4 patient cells, supporting the idea of SETX being a downstream target of ZPR1 (FIGS. 9E and 9F).

Further, IP of R-loops from normal and ALS4 cells overexpressing ZPR1 shows increase in in vivo binding of SETX with R-loops compared to control cells (FIG. 9G). Quantitation of SETX levels Co-IP with R-loops shows significant improvement in the binding of SETX from (34.24±4.30%) to (90.03±11.39%, P=0.0101) in ALS4 cells with ZPR1 overexpression (ALS4+ZPR1-GFP) compared to control (ALS4+GFP) cells (FIG. 9H). These data suggest that ZPR1 has the potential to regulate SETX levels under normal and ALS4 conditions and rescues pathogenic R-loop phenotype in ALS4 patient cells. Together, these findings provide insight into the function of ZPR1-SETX complexes in R-loop resolution and indicate the disruption of ZPR1-SETX complexes as the molecular basis for ALS4 pathogenesis.

### Example 1.15. Discussion

In this Example, Applicant provides insight into the molecular basis of R-loop resolution and the pathogenesis of ALS4. Applicant uncovered the critical role of SETX-ZPR1 complexes in R-loop resolution and identify the putative function of ZPR1 as a molecular brake to regulate SETX-dependent R-loop resolution activity. Overall, findings of this Example allowed Applicant to delineate the molecular mechanism of R-loop resolution under the normal and ALS4 conditions. Applicant demonstrate that ZPR1 forms endogenous complexes with SETX and R-loops. ZPR1 is critical for recruiting SETX onto R-loops and is required for the resolution of RNA-DNA hybrids formed during transcription. The observation that SETX interacts with ZPR1 and shows highest colocalization (˜95%) with ZPR1⁺ nuclear bodies compared to SMN⁺ (gems) (˜70%) and coilin+(Cajal bodies) (˜55%), indicates that these proteins may function jointly. Moreover, because ZPR1 is required for the recruitment of SETX onto R-loops, ZPR1 may be involved in the physiological functions of SETX. Applicant's data demonstrates that low levels of SETX-ZPR1 complexes result in the loss-of-function leading to an increase in R-loop levels and motor neuron degeneration in SMA.

SETX mutation (L389S) disrupts SETX-ZPR1 complexes, which results in gain-of-function leading to fewer R-loops and motor neuron degeneration in ALS4. Together, these findings originate a novel concept in the field of cell and molecular biology, opposite alterations in a cell biological activity (R-loop resolution) result in similar pathogenesis (neurodegeneration) in different genetic motor neuron disorders.

Findings of this study unravel the key steps that may be a part of the molecular mechanism of R-loop resolution under normal and ALS4 disease conditions. Applicant's data demonstrate that following key steps may contribute to R-loop resolution: (i) ZPR1 tethers to RNA-DNA hybrids, (ii) recruits SETX onto R-loops and (iii) may function as a “molecular brake” to regulate SETX-dependent RLRC activity. Mutation in SETX disrupts its interaction with ZPR1, which may cause partial impairment of the molecular brake, resulting in higher activity of R-loop resolution (gain-of-function) leading to fewer R-loops in ALS4 compared to control.

ZPR1 is essential for cell viability. However, the physiological function of ZPR1 that is critical for cell survival is unknown. Here, Applicant shows that ZPR1 deficiency causes downregulation of SETX, suggesting that SETX is likely a downstream target of ZPR1. Accumulation of R-loops throughout gene transcription in ZPR1-deficient cells suggests that ZPR1-deficiency may primarily impair R-loop resolution while transcription continues and R-loop-mediated DNA damage causes genomic instability leading to neurodegeneration and cell death. These findings suggest that ZPR1 is critical for resolution of co-transcriptional R-loops and contributes to one of the fundamental cellular processes essential for cell viability.

Diversion from normal R-loop levels is linked to neurodegenerative disorders such as ALS4 (low R-loops) and SMA (high R-loops). It is intriguing that contrasting levels of R-loops result in a common phenotype of motor neuron degeneration in two genetic diseases, ALS4 and SMA, caused by mutations in the SETX and SMN1 genes, respectively. Applicant's data demonstrate that ALS4 and SMA patient cells have a similar defect, which is ˜67˜70% decrease in SETX association with R-loops but opposite cellular phenotype with low and high levels of R-loops, respectively. Comparison of SMA and ALS4 patient cells data reveals key molecular differences that may explain these contrasting as well as fascinating cellular phenotypes.

In SMA, expression of ZPR1 and SETX are downregulated, which results in low levels of ZPR1-SETX complexes. Downregulation of ZPR1 and SETX is likely the consequence of global splicing defects caused by SMN deficiency in SMA. In addition, defects in splicing can alter transcription because of interdependence between transcription and splicing and may contribute to R-loop accumulation. The decreased levels of ZPR1-SETX complexes impair the efficiency of R-loop resolution (loss-of-function) and result in higher levels of R-loops in SMA. This is supported by the observations that the knockdown of either ZPR1 or SETX results in low levels of ZPR1-SETX complexes and causes R-loop accumulation.

On the other hand, the levels of ZPR1 and SETX are not altered in ALS4. However, mutation (L389S) in SETX abolishes its interaction with ZPR1, which impairs ZPR1 ability to recruit mutant SETX* to R-loops resulting in decreased levels of SETX onto R-loops but results in fewer R-loops in ALS4. In addition, the observation that the mutation in SETX disrupts interaction with ZPR1 but does not affect ZPR1 binding with R-loops in ALS4 suggests that ZPR1 can bind to R-loops independently of SETX and supports the idea that ZPR1 binds first and then recruits SETX to R-loops. Therefore, the disruption of ZPR1-SETX complexes may be the cause of increase in R-loop resolution activity leading to fewer R-loops in ALS4.

Applicant proposes that ZPR1 collaborates with SETX and functions as a molecular brake to regulate SETX-dependent R-loop resolution activity. It is possible that ZPR1-SETX-SETX* (ALS4) complexes possess hyper-activity compared to ZPR1-SETX-SETX-ZPR1 (normal) complexes because of the partial impairment of the molecular brake resulting in faster R-loop resolution leading to fewer R-loops in ALS4 compared to normal. Applicant's additional data further support the proposed model and also demonstrate that SETX is a downstream target of ZPR1.

Knockdown of ZPR1 causes downregulation of SETX expression, which result in accumulation of R-loops in ALS4 patient cells, similar to SMA cells, suggesting that ZPR1-dependent activity is also critical for R-loop resolution in ALS4 cells. Notably, overexpression of ZPR1 increases SETX levels, improves recruitment of SETX onto R-loops and rescues pathogenic R-loop phenotype in ALS4 patient cells. These data suggest that increasing the ratio of normal ZPR1-SETX-SETX-ZPR1 versus mutant ZPR1-SETX-SETX* (ALS4) complexes can rescue pathogenic R-loop phenotype in ALS4 and (ii) ZPR1 may represent a potential molecular target that could be exploited therapeutically. These data suggest that ZPR1 may be a potential regulator of SETX-dependent R-loop resolution activity.

Applicant's study also provides insight into the mechanism of predominant degeneration of motor neurons in patients with ALS4 and SMA. Applicant's data demonstrates that ZPR1 regulates expression of SETX levels. In SMA, chronic low levels of ZPR1 may contribute to downregulation of SETX. Decrease in ZPR1-SETX complexes results in R-loop accumulation that causes DSBs. SMA motor neurons express low levels of DNA-PKcs, which is required for NHEJ-mediated DNA repair, the primary DSB repair mechanism available in post-mitotic neurons. Deficiency of DNA-PKcs may impair DNA repair in motor neurons leading to genomic instability and neurodegeneration in SMA. Increase in ZPR1 levels resulted in two-pronged improvement in SMA. ZPR1 overexpression restores SETX levels and improves assembly and activity of RLRC, reducing R-loop accumulation in SMA. Furthermore, ZPR1 increases DNA-PKcs levels and rescues DNA damage in neurons, preventing neurodegeneration in SMA. These data suggest that genomic instability may be the cause of selective degeneration of motor neurons in SMA.

ALS4 is caused by heterozygous mutation in the SETX gene and characterized by motor neuron degeneration and neuromuscular weakness. However, the molecular basis of pathogenesis caused by the mutant SETX protein was unclear. Applicant provides insight into the molecular events altered by mutant SETX, which contribute to pathogenic low levels of R-loops causing neurodegeneration in ALS4. Applicant's data demonstrate that disruption of SETX interaction with ZPR1 caused by the SETX mutation (L389S) may be a cause of increase in R-loop resolution activity leading to fewer R-loops in ALS4. Low levels of R-loops are shown to decrease the expression of BMP and activin membrane bound inhibitor (BAMBI), a negative regulator of transforming growth factor-β (TGF-β) pathway, in cells derived from ALS4 patients.

BAMBI binds to TGF-β receptor and blocks the interaction of TGF-β with receptors, and negatively regulating signaling. The TGF-β pathway plays an important role in survival and axon guidance of motor neurons and in the pathogenesis of ALS. Therefore, mutant SETX-mediated decrease in R-loop levels and R-loop-dependent downregulation of BAMBI may cause pathogenic increase in TGF-β signaling and contribute to motor neuron degeneration in ALS4. In addition, mislocalization of nuclear TAR DNA binding protein (TARDBP or TDP-43) in the cytoplasm of ALS4 patient spinal cord motor neurons may also contribute to neurodegeneration in ALS4 through a common pathogenic mechanism involved in ALS caused by mutations in TDP-43

Currently, there is no treatment available for a growing number of incurable disorders caused by defects in R-loop metabolism. The observation that ZPR1 can regulate R-loop accumulation and rescue pathogenic R-loop phenotypes in SMA and ALS4 patient cells suggest that ZPR1 may be a potential therapeutic target for manipulating R-loop levels and for developing treatments for diseases with altered R-loop metabolism, including ALS4.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of treating or preventing a disorder in a subject, said method comprising:
administering to the subject a composition, wherein the composition comprises at least one active component selected from the group consisting of:
a zinc finger protein ZPR1 (ZPR1), an analog thereof, a homolog thereof, a derivative thereof, or combinations thereof;
an enhancer of expression of ZPR1, an analog thereof, a homolog thereof, or combinations thereof;
nucleotides encoding ZPR1, an analog thereof, a homolog thereof, a derivative thereof, or combinations thereof; or
combinations thereof,
wherein the disorder is a neurodegenerative disease or disorder, a disease or disorder caused by at least one mutation in the Senataxin (SETX) gene, a disease or disorder caused by downregulation of SETX protein levels, or combinations thereof.

2. The method of claim 1, wherein the active component comprises ZPR1.

3. The method of claim 2, wherein ZPR1 is represented by a peptide sequence comprising SEQ ID NO: 1, or a peptide sequence sharing at least 65% sequence homology to SEQ ID NO: 1.

4. The method of claim 1, wherein the active component comprises an analog or homolog of ZPR1, wherein the analog or homolog is at least 80% identical in peptide sequence to ZPR1.

5. (canceled)

6. The method of claim 1, wherein the active component comprises a derivative of ZPR1.

7. The method of claim 6, wherein the derivative comprises a recombinant ZPR1 fused to a green fluorescent protein (ZPR1-GFP).

8. The method of claim 1, wherein the active component comprises a nucleotide sequence encoding ZPR1.

9. The method of claim 8, wherein the nucleotide sequence is in the form of DNA.

10. The method of claim 8, wherein the nucleotide sequence is in the form of mRNA.

11. The method of claim 8, wherein the nucleotide sequence comprises SEQ ID NO: 2, or a nucleotide sequence sharing at least 65% sequence homology to SEQ ID NO: 2.

12. The method of claim 8, wherein the nucleotide sequence comprises a mRNA transcript of SEQ ID NO: 2, or a mRNA transcript of a nucleotide sequence sharing at least 65% sequence homology to SEQ ID NO: 2.

13. The method of claim 1, wherein the active component comprises a derivative of a nucleotide sequence encoding ZPR1.

14. The method of claim 13, wherein the derivative comprises a nucleotide sequence encoding ZPR1 fused to a nucleotide sequence encoding a green fluorescent protein (ZPR1-GFP).

15. The method of claim 1, wherein the administering comprises intravenous administration, subcutaneous administration, transdermal administration, topical administration, intraarterial administration, intrathecal administration, intracranial administration, intraperitoneal administration, intraspinal administration, intranasal administration, intraocular administration, oral administration, or combinations thereof.

16. The method of claim 1, wherein the disorder comprises a disease or disorder caused by at least one mutation in the Senataxin (SETX) gene.

17. The method of claim 1, wherein the disorder comprises a neurodegenerative disease or disorder, wherein the neurodegenerative disease or disorder is selected from the group consisting of amyotrophic lateral sclerosis 4 (ALS4), ataxia with oculomotor apraxia type 2 (AOA2), spinal muscular atrophy (SMA), autosomal dominant SMA (ADSMA) or combinations thereof.

18-19. (canceled)

20. The method of claim 1, wherein the administered or expressed ZPR1 treats or prevents the disorder by binding to co-transcriptional RNA-DNA hybrids (R-loops), recruiting Senataxin (SETX) onto the R-loops, and regulating the prevalence of the R-loops, wherein the regulating comprises decreasing or increasing R-loop accumulation.

21-22. (canceled)

23. The method of claim 20, wherein the disorder to be treated or prevented comprises spinal muscular atrophy (SMA), autosomal dominant SMA (ADSMA), or combinations thereof, and wherein the administered or expressed ZPR1 treats or prevents the SMA by decreasing R-loop accumulation.

24. The method of claim 20, wherein the disorder to be treated or prevented is amyotrophic lateral sclerosis 4 (ALS4), and wherein the administered or expressed ZPR1 treats or prevents the SMA by increasing R-loop accumulation.

25. The method of claim 1, wherein the subject is selected from the group consisting of a mammal, a human being, or combinations thereof.

26-50. (canceled)


SEQUENCE LISTING (2 sequences)

SEQ ID NO: 1 - DNA, 1380 bp, Artificial Sequence
  Human ZPR1 cDNA
atggcggccagcggggctgtggaaccagggcccccgggggctgccgtcgccccgtcgcccgccccggccccgccgcctgcccctgatcacctgttccggcccatcagcgccgaggacgaggagcagcagcccaccgagatcgagtcgctatgcatgaactgttactgcaatggcatgacgcgcctcctgctcaccaagattcccttcttcagagaaataatagtgagctccttttcctgcgagcactgtggctggaacaacacggagatccagtcggcaggcaggatccaggaccagggagtgcgctacactttgtctgtcagggctctggaggacatgaacagagaagtggtgaagactgactctgctgccacaaggattcctgagctagattttgaaattcctgcctttagccagaaaggagctctgaccactgttgaaggattgatcacccgtgctatctctggcctggagcaggaccagcctgcacgaagggcaaacaaagatgctacagctgaaagaattgatgagttcattgtcaaactgaaggagctaaagcaagtagcctcccctttcactctgatcattgatgatccctcagggaacagttttgtggaaaacccacatgctcctcagaaagatgatgccctggtgatcacacactacaaccggacccgacagcaggaagagatgctggggcttcaagaagaagcaccagcagagaagccagaagaggaagatctcagaaatgaagtgctccagttcagcacaaactgcccagaatgcaatgcccccgctcagaccaacatgaagctagtacaaatccctcactttaaggaggttatcatcatggctaccaactgcgagaactgtgggcatcggaccaatgaggtgaaatctggaggagcagtagaacccttgggcaccaggatcaccctccacatcacagatgcctcagatatgaccagagacctcctcaagtctgagacttgcagtgtggaaatcccagagctagaatttgaactgggaatggcagtcctcgggggcaagttcaccacactggaagggctgctgaaagacatccgggaactggtgaccaaaaatcctttcacactgggcgacagttccaatcctggacagacggagagactacaggagtttagccagaagatggaccagatcatcgaaggtaacatgaaggcccactttattatggatgatccagcaggaaacagttacttgcagaatgtgtatgcgcctgaagatgatcctgagatgaaggtggagcgttacaagcgcacctttgaccaaaatgaggagctagggctcaatgacatgaagacagagggctatgaggcaggcctggctccgcaacggtag

SEQ ID NO: 2 - protein, 459 aa, Artificial Sequence
  Human ZPR1 Protein
MAASGAVEPGPPGAAVAPSPAPAPPPAPDHLFRPISAEDEEQQPTEIESLCMNCYCNGMTRLLLTKIPFFREIIVSSFSCEHCGWNNTEIQSAGRIQDQGVRYTLSVRALEDMNREVVKTDSAATRIPELDFEIPAFSQKGALTTVEGLITRAISGLEQDQPARRANKDATAERIDEFIVKLKELKQVASPFTLIIDDPSGNSFVENPHAPQKDDALVITHYNRTRQQEEMLGLQEEAPAEKPEEEDLRNEVLQFSTNCPECNAPAQTNMKLVQIPHFKEVIIMATNCENCGHRTNEVKSGGAVEPLGTRITLHITDASDMTRDLLKSETCSVEIPELEFELGMAVLGGKFTTLEGLLKDIRELVTKNPFTLGDSSNPGQTERLQEFSQKMDQIIEGNMKAHFIMDDPAGNSYLQNVYAPEDDPEMKVERYKRTFDQNEELGLNDMKTEGYEAGLAPQR